(19) United States
(12) Patent Application Publication
(10) Pub. No.: US 2023/0129485 A1
(43) Pub. Date: Apr. 27, 2023

(54) SYSTEM AND METHOD FOR DETERMINING MATERIAL PROPERTIES OF MOLECULAR SYSTEMS FROM AN AB-INITIO PARAMETERIZED FORCE-FIELD

(71) Applicant: InterX, Inc., Berkeley, CA (US)

(72) Inventors: Leonid PEREYASLAVETS, Lafayette, CA (US); Boris FAIN, Berkeley, CA (US); Ganesh KAMATH, El Sobrante, CA (US)

(21) Appl. No.: 17/967,451

(22) Filed: Oct. 17, 2022

Related U.S. Application Data
(60) Provisional application No. 63/271,979, filed on Oct. 26, 2021.

(51) Int. Cl. G16C 10/00; G16C 20/50; G16C 20/70

(52) U.S. Cl. CPC G16C 10/00; G16C 20/50; G16C 20/70

(57) ABSTRACT

In some embodiments, a method includes determining an atom type of each atom from a set of atoms in a functional group of a molecular system. The method includes calculating, based on the set of atom types, a first set of ab initio molecular properties of a monomer and a second set of ab initio molecular properties of a multimer. The method further includes determining a set of parameters of a force field model by fitting the force field model to the first set of ab initio molecular properties and the second set of ab initio molecular properties until at least an agreement reaches a pre-determined threshold. The method includes determining, based on the set of parameters and the force field model, a molecular property of the molecular system and sending a signal to present the molecular property of the molecular system.

### CROSS-REFERENCE TO RELATED APPLICATION

This application claims priority to and the benefit of U.S. Provisional Patent Application No. 63/271,979 filed Oct. 26, 2021, and entitled “System and Method for Determining Material Properties of Molecular Systems from an ab-initio Parameterized Force-Field,” the contents of which are incorporated herein by reference in its entirety.

### TECHNICAL FIELD

Some embodiments described herein relate to computer-implemented methods and systems of determining material properties of molecular systems. In particular, but not by way of limitation, some embodiments described herein relate to computer-implemented methods and systems of determining material properties of molecular systems from an ab-initio parameterized force-field.

### BACKGROUND

Molecular simulation is used to predict experimental observables of molecular systems. A force field model of a molecular system is a representation of the energy of the system (i.e., the Hamiltonian) which can be used to determine properties of a physical system. However, devising interaction models or force field (FF) models for arbitrary molecules with little or no reliance on experimental data has presented challenges. In addition, some known molecular force field models and molecular simulation methods either do not extend to a wide coverage of molecular systems or fail to determine properties within an acceptable degree of chemical accuracy.

Accordingly, a need exists for a wide coverage force field model and a molecular simulation method that are transferable and extendable to previously un-benchmarked chemical species, have minimum reliance on experimental data, and predict experimentally observable properties within an acceptable degree of chemical accuracy.

### SUMMARY

In some embodiments, a method includes determining, by a processor, an atom type from a set of atom types of each atom from a set of atoms in a functional group of a molecular system. The method includes calculating, by the processor and based on the set of atom types, a first set of ab initio molecular properties of a monomer having the functional group and a second set of ab initio molecular properties of a multimer having the functional group. The method further includes determining, by the processor, a set of parameters of a force field model by fitting the force field model to the first set of ab initio molecular properties and the second set of ab initio molecular properties until at least an agreement reaches a pre-determined threshold. The agreement can include one of (1) a first difference between a first set of predicted molecular properties of the monomer and the first set of ab initio molecular properties of the monomer or (2) a second difference between a second set of predicted molecular properties of the multimer and the second set of ab initio molecular properties of the multimer. The method includes determining, by the processor and based on the set of parameters and the force field model, a molecular property of the molecular system and sending, by the processor, a signal to present the molecular property of the molecular system.

Embodiments described herein include a parameterized force field model and a parameterized force field molecular simulation system and/or method using the parameterized force field model. The parameterized force field model can be transferable, have wide coverage, and have the property of extending interactions to previously un-benchmarked chemical species. The parameterization of the force field molecular simulation method discussed herein can be based on first principles that have little or no reliance on experimental data. Embodiments described herein can predict experimentally observable properties of molecular systems within an acceptable degree of chemical accuracy.

### BRIEF DESCRIPTION OF THE DRAWINGS

FIG. 1 illustrates an example schematic diagram illustrating the QM-parameterized force-field computing device, according to some embodiments.

FIG. 2 shows the density-inspired description of interatomic and intermolecular energies and forces, as well as the shift of electron density under external forces (polarization), according to some embodiments.

FIG. 3 shows a flow chart illustrating an example QM-parameterized force-field molecular simulation process, according to some embodiments.

FIGS. 4A-4B show flow charts illustrating an example QM-parameterized force-field molecular simulation process, according to some embodiments.

FIGS. 5A-5G illustrate the correspondences and deviations between the QM calculations and force field model predictions, according to some embodiments.

FIG. 6 shows the non-additive many-body error of select optimized water multimers in relation to the total QM intermolecular energy, according to some embodiments.

FIGS. 7A and 7B show the agreement between the force field predictions and experiment hydration of anthracene, according to some embodiments.

FIG. 8 shows the predictions of radial distribution function (RDF) of oxygen-oxygen (O—O) distance in water, using the QM-parameterized force field model, according to some embodiments.

FIGS. 9A-9C show solvation and hydration free energies predictions of various solutes, using the QM-parameterized force field model, according to some embodiments.

FIGS. 10A-10B show predicted and experimental results of the hydration including predicted results using the QM-parameterized force field model, according to some embodiments.

FIG. 11 shows a double logarithm learning plot constructed with a subset of the total set of dimers, according to some embodiments.

### DETAILED DESCRIPTION

A force field is a method that can be used to estimate the forces between atoms within molecules and also between molecules. The force field refers to the functional form and parameter sets used to calculate the potential energy of a system of atoms or coarse-grained particles in molecular mechanics, molecular dynamics, or Monte Carlo simulations. The parameters for a chosen energy function can be derived or adjusted from experiments in physics and chemistry, calculations in quantum mechanics, or both.

The force field parameters and interaction rules in applications to molecular systems can define the energy landscape from which a number of results of interest can be derived. For example, the acting forces on particles can be derived as a gradient of the potential energy with respect to the particle coordinates. For another example, the resulting equations of motion can be propagated forward in time and time averages can predict experimental observables.

Prediction of experimentally observable ensemble and non-ensemble properties of a material system may be obtained from a molecular force field model of the material system. An ensemble property may be obtained via molecular simulation of the model, for example, molecular dynamics (MD), path integral molecular dynamics (PIMD), or Monte Carlo (MC). The way to determine the ensemble properties from the Hamiltonian at non-zero temperature can be, for example, via various thermodynamic sampling methods such as molecular dynamics (MD), path integral molecular dynamics (PIMD), or Monte Carlo (MC).

The molecular interaction models can be roughly separated into two categories: scope limited models that benchmark their interaction energies with Quantum Mechanics (QM) calculations, and wide coverage models that fit at least some of their parameters to selected sets of experimental data. Some known QM parametrized models can employ high functional complexity to reproduce QM energies. The functional complexity of these known QM-parameterized models, however, can make transferability and extending coverage to other chemical species and hetero-chemical ensembles difficult.

Some known wide-coverage molecular force field models derive some or all of the parameters by fitting to empirical observables. There are at least two drawbacks to this approach. First, even available experimental data (e.g., densities, heats of vaporization) can be insufficient to produce models that describe existing compounds precisely; and there can be molecules (for example, molecules that have not been synthesized) that require more precise description than available data from existing inference. Second, if an empirical model's prediction is erroneous, it can be difficult to decide which parameter(s) of the model to remove, add, correct or adjust.

In a wide coverage force field, a chemical group can be considered described when both its self-interactions and its interactions with other described group(s) are determined. A Force Field with wide coverage can, in some examples, fully describe the interactions between most and/or all chemical groups. In the case of a protein, because of the importance of biology, wide coverage force fields can include the interaction of amino acids and backbone, both between themselves, water, and non-polar solvents at the very minimum. Furthermore, in some instances, the force field can describe the interactions between a significant number of major chemical groups, e.g., alkanes aromatics amides amines sulfides esters, in both homo (self) and hetero (between any distinct number of) interactions. More specifically, in some instances, the force field can describe the behavior of these groups in a variety of solvents: H2O, CHEX (cyclohexane), OCT (octanol), Benzene, etc.

Finally the simulation results produced with the force-field can correctly describe the entropic behavior of major solvents—H2O, CHEX, OCT, benzene, in order to capture the entropic component of solvation. Moreover the results can correctly describe the entropic behavior of arbitrary molecular systems.

Some known fixed charge force field models lack flexibilities in the functional form to fit the experimental results well and frequently the errors are uncontrollable. The parameters of these known fixed charge force field models that are determined by QM energies are not well defined, as these models do not have polarization, and are, therefore, unable to transfer the results obtained by QM (in the gas phase) into the liquid phase or the solid phase (condensed phase). Furthermore, when fitting to experimental results, these known models do not have clear guidance on which parameters should be corrected, and therefore errors also arise.

The known Merck Molecular Force Field (MMF94) model, for example, also lacks polarization and includes ad-hoc factors. The functional form of the MMF94 model does not permit transferability from the available QM calculations to larger collections of molecules, e.g., to the condensed phase. As a result, the known MMF94 model produces unacceptable low quality predictions and does not enable accurate molecular modelling predictions from first principles.

Some known machine learning models lack the precision, transferability or coverage for describing arbitrary molecular ensembles. In addition, these known machine learning models have not been incorporated into a fully functional simulation stack with, e.g., long distance truncation, nuclear quantum effects, or thermodynamic property outputs.

Embodiments described herein include a QM-parameterized force field model and a molecular simulation method, which include the benefits of: (1) interaction energy calculated from first principles and have little, minimum or no reliance on experimental data, (2) transferable and extendable to other molecular systems, and (3) predicts experimentally observable properties within an acceptable degree of chemical accuracy. The QM-parameterized force field models and the molecular simulation methods described herein can accurately predict experimental observables of molecular systems with models for arbitrary molecules with little, minimum or no reliance on experimental data.

Example embodiments are illustrated in the drawings. It is to be understood that the embodiments are described only to facilitate better understanding of the embodiments, rather than to limit the scope of the embodiments in any manner.

An advantage of the QM-parameterized force field models described herein is that in some embodiments, QM calculations can be obtained for arbitrary molecules. In some embodiments, a limitation may exist on QM computations of the size of the molecular system. Another advantage of the QM-parameterized force field models described herein is that prediction errors can be traced to, for example, the imprecise description of the interaction energies and rectified in the model. In some implementations, embodiments described herein include methods to generate models parameterized entirely from first-principles (ab-initio) Quantum Mechanics calculations.

The QM-parameterized force field models described herein can be multipolar, polarizable, and the dynamics of the QM-parameterized force field models can include nuclear quantum effects. The QM-parameterized force field models and the molecular simulation methods described herein can maintain a high computational efficiency. The construction of a wide-coverage molecular model from first principles and its excellent predictive ability in the liquid phase, as described in embodiments herein, is an advance in molecular simulation.

Embodiments described herein include an ab-initio-based molecular interaction model with chemical accuracy that enables the quantitative in-silico determination of properties of molecular ensembles.

Embodiments described herein include QM-parameterized force field models applicable to arbitrary molecules. The QM-parameterized force field models can predict solvation free energies of molecular systems within chemical accuracy (in some implementations, better than thermal noise at room temperature (˜0.59 kcal/mol)). In some implementations, the predictions in the liquid phase can be satisfyingly accurate while the model is created from ab-initio computational methods without fitting to any experimental data. The value of 0.5 kcal/mol for the desired (‘chemical’) accuracy of free energy predictions arises from several considerations. First, 0.59 kcal/mol is the thermal noise at ambient conditions (room temperature and pressure). This is the inherent fuzziness of our everyday biological world. Additionally, for example, in ligand-protein lead optimization the definition of ‘incremental lead improvement’ is about 0.5 kcal/mol or ˜2-3 fold increase binding affinity.

In some implementations, the QM-parameterized force field models described herein can include a set of features that enables the final energies of the QM-parameterized force-field models to agree with the final energies of a small collection of molecules within an average chemical accuracy below 0.5 kcal/mol. In some implementations, for neural-neutral chemical groups in a molecular system, such chemical accuracy can be improved to around 0.3 kcal/mol. In some implementations, for some neural-charged proper ions in a molecular system, such chemical accuracy can be worsened to be higher than 0.5 kcal/mol. The set of features can include, for example, (1) fully multipolar which can provide accurate fitting, (2) polarizable, (3) model parameters that are assigned to interaction centers (i.e., atoms) and intramolecular degrees of freedom (i.e., bond stretch energies), (4) model parameters that depend on and are changed by different chemical (i.e., within a molecule) molecular environments, and/or (5) transferability.

In some implementations, the QM-parameterized force-field molecular simulation methods described herein can extend and combine models for sub-pieces of a molecule when, for example, the whole molecule(s) are too large to compute.

In some implementations, the QM-parameterized force-field molecular simulation methods can provide a certain accuracy by including specific algorithms and/or processes in the downstream simulation protocols. One set of such algorithms can be, for example, a proper truncation method of long-range interactions (e.g. electrostatic and dispersion). A specific example of such a truncation method can be the Particle Mesh Ewald method (PME). Another set of such algorithms can be, for example, methods that may permit one to include quantum mechanics effects. A specific example of these algorithms may be described as Path Integral Molecular Dynamics (PIMD).

In some embodiments, Molecular Mechanics (MM) can refer to a Newtonian representation of molecular interaction, where the energy is a function of atomic and molecular geometries, distances and orientations. This includes monomer, two-body and n-body contributions.

In some embodiments, Quantum Mechanics (QM) can refer to a quantum mechanical description and propagation of the molecular system. It may mean an N-body wavefunction solution, or DFT (Density Functional Theory), which reduces computational load by using an electron density functional instead of an n-body wavefunction.

In some embodiments, transferable can refer to the property of extending interactions to previously un-benchmarked chemical species. For a transferrable interaction, having determined such between A<->B and B<->C, the interactions between A<->C are automatically determined and correct. The electrostatic interaction is an example.

In some embodiments, separable can refer to an interaction where the properties of the interaction between species are a function of properties belonging to the individual involved species. For example, an electrostatic interaction is proportional to the product of the individual charges q1*q2.

In some embodiments, a combination rule can be the actual function giving the energy of a separable interaction from individual properties, e.g., q1*q2/r.

The parameterized force field model, the QM-parametrized force field model, the QM-parameterized model, and the QM-parameterized physics-based molecular models can be used interchangeably in embodiments described herein.

In some embodiments, the “interaction rank”, L, can indicate the nature of interaction between two multipole moments. In some implementations, when L=0, monopole-monopole interactions are considered, while L=2 can mean a dipole-dipole interaction or a monopole-quadrupole interaction is considered.

In some embodiments, chemical accuracy may refer to thermal noise at room temperature and pressure which is 0.59 kcal/mol or kT at 300K.

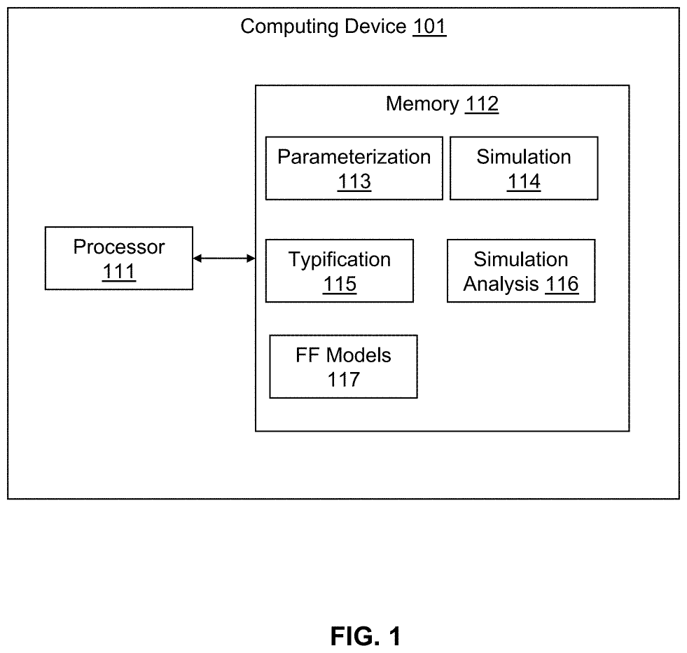

Embodiments described herein that relate to dimer(s) can also apply to multimer(s). In some implementations, a multimer can be a molecule that includes two or more monomers. In other words, a multimer can be a dimer, a trimer, a tetramer, pentamer, hexamer, and so on. For example, the QM-parameterized force field computing device (e.g., 101 in FIG. 1) can be configured to calculate a set of ab initio molecular properties of a monomer having a functional group and a set of ab initio molecular properties of a multimer (e.g., a dimer) having the functional group. The QM-parameterized force field computing device (e.g., 101 in FIG. 1) can predict a set of molecular properties of the monomer and a set of molecular properties of the dimer.

### System

FIG. 1 illustrates an example schematic diagram illustrating the QM-parameterized force-field computing device 101, according to some embodiments. The QM-parameterized force-field computing device 101 can be a compute device (or multiple compute devices) having a processor 111 and a memory 112 operatively coupled to the processor 111. In some instances, the QM-parameterized force-field computing device 101 can be any combination of hardware-based component (e.g., a field-programmable gate array (FPGA), an application specific integrated circuit (ASIC), a digital signal processor (DSP), a graphics processing unit (GPU)) and/or software-based component (computer code stored in memory 112 and/or executed at the processor 111) capable of performing one or more specific functions associated with that component. In some instances, the QM-parameterized force-field computing device 101 can be a server such as, for example, a web server, an application server, a proxy server, a telnet server, a file transfer protocol (FTP) server, a mail server, a list server, a collaboration server and/or the like. In some instances, the QM-parameterized force-field computing device 101 can be a personal computing device such as a desktop computer, a laptop computer, a personal digital assistant (PDA), a mobile telephone, a tablet personal computer (PC), and/or so forth.

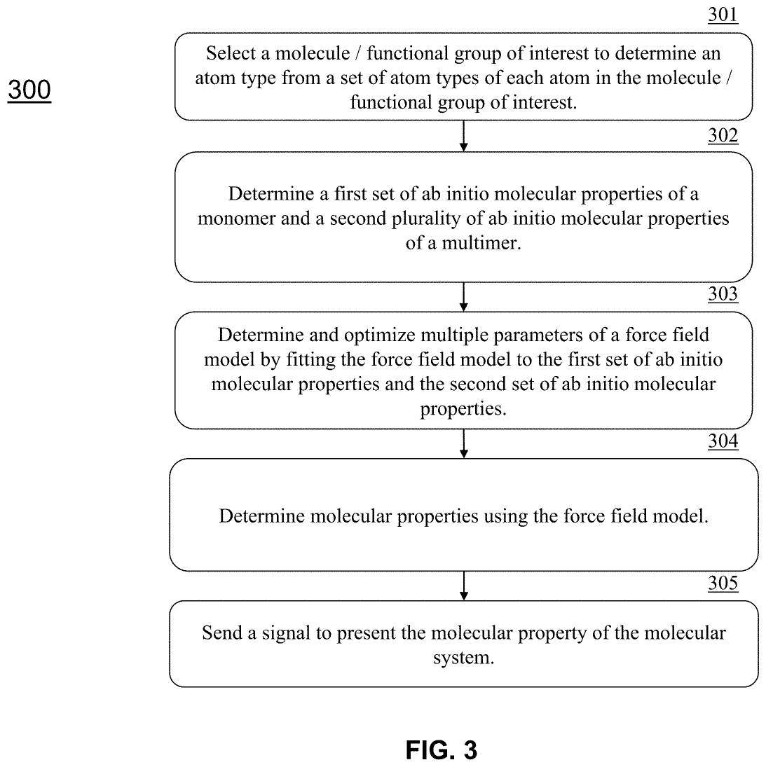

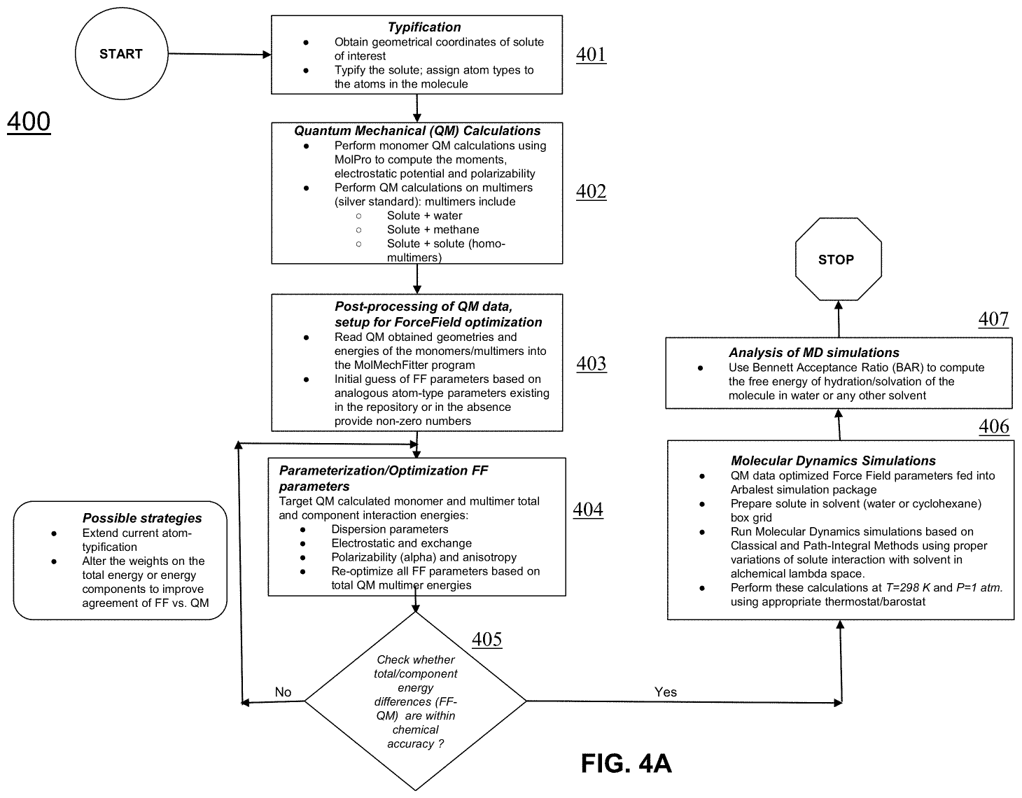

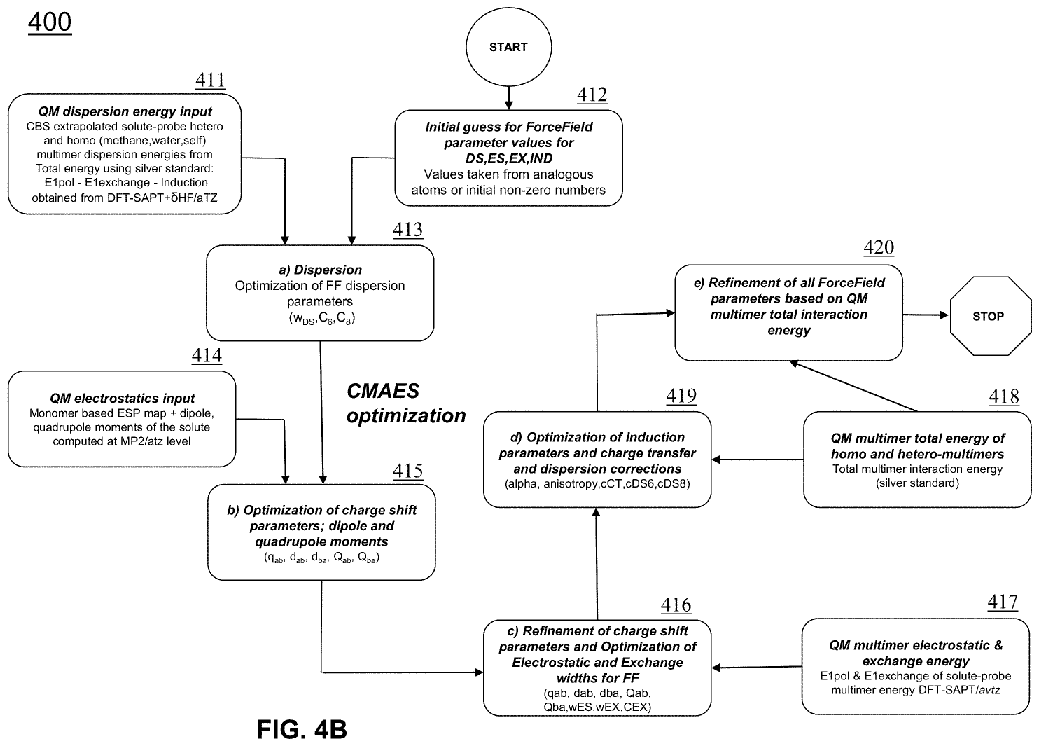

The memory 112 can be, for example, a random-access memory (RAM) (e.g., a dynamic RAM, a static RAM), a flash memory, a removable memory, a hard drive, a database and/or so forth. In some implementations, the memory 112 can include (or store), for example, a database, process, application, virtual machine, and/or other software modules (stored and/or executed in hardware) and/or hardware modules configured to execute a QM-parameterized force-field molecular simulation process(es) as described with regards to FIG. 3 and FIGS. 4A-4B. In such implementations, instructions for executing the parameterized force-field molecular simulation process(es) and/or the associated methods can be stored within the memory 112 and executed at the processor 111.

The processor 111 can be configured to, for example, write data into and read data from the memory 112, and execute the instructions stored within the memory 112. The processor 111 can also be configured to execute and/or control, for example, the operations of other components of the parameterized force-field computing device 101 (such as a network interface card, other peripheral processing components (not shown)). In some implementations, based on the instructions stored within the memory 112, the processor 111 can be configured to execute one or more steps of the QM-parameterized force-field molecular simulation process(es) as described with regards to FIG. 3 and FIGS. 4A-4B.

In some implementations, the processor 111 can be configured to execute a QM-parameterized force-field molecular simulation method that can include, for example, the steps: (1) creating a QM description of the atoms of a molecular system with required accuracy; (2) creating a force field model from the QM description; (3) optionally, extending or transferring the existing force field model to new atom types; and (4) performing thermodynamic sampling with a simulation method such as, for example, MD or MC, that incorporates procedures such as nuclear quantum effects (NQE) to obtain results, i.e., ensemble properties that predict experimentally observable properties with chemical accuracy.

In some implementations, the memory 112 may include a parameterization software stack 113, a simulation software stack 114, a typification stack 115, a simulation analysis stack 116, and force field models 117. Each of the parameterization software stack 113, the simulation software stack 114, the typification stack 115, and the simulation analysis stack 116 can include code having instructions that, when executed by the processor 111, cause the processor 111 to perform at least a portion of the steps in the QM-parameterized force-field molecular simulation process. In some implementations, the force field models 117 and information of the force field models 117 can be stored in, for example, flat files, in suitable markup language files, in memory, in a database format, etc. The formats can be human or machine readable. In some implementations, the memory 112 can include a database that can store the interaction parameters of the QM-parameterized force field model and the processor 111 can update, manage, and control access to the database. The processor 111 can also perform consistency and integrity checks of the data stored in the database when, in some implementations, the data are added to the database. For example, when adding information of molecular properties or interaction parameters of a new non-bonded type of molecules, information of a corresponding bonded type of the molecules may be needed and/or used for the QM-parameterized force-field molecular simulation process. In such situations, the processor 111 can automatically send a notification (to, for example, a compute device of a user) to request such information to be added to the database. In some implementations, the interaction parameters provided by the database for the QM-parameterized force-field molecular simulation process may be protected by encryption. The processor 111 may assign different access rights to users to have different levels of decryption and/or access capabilities. For example, a first user can access to a first set of interaction parameters of the QM-parameterized force field model, which is not accessible by a second user. Thus, the QM-parameterized force-field molecular simulation process performed for the first user can be kept confidential from the QM-parameterized force-field molecular simulation process performed for the second user.

The typification software stack 115, when executed by the processor 111, can be configured to determine an atom ‘type’ of an atom based on the atom's QM monomer properties and the atom's chemical environment in the molecule. An atom ‘type’ can be a classification of an atom, which can depend on its atomic number. An atom ‘type’ can also be further differentiated based on its chemical connections to other atoms in the molecule and can help the interaction parameters to better represent such subclasses. An example of such differentiating atom types can be an aromatic carbon and an aliphatic carbon.

The simulation software stack 114 can include, for example, an MD package which can contain features designed to support the multi-featured QM-parameterized force field model. For example, the features can include, but are not limited to, multipolar exchange interactions. In some implementations, these features (e.g., multipolar exchange interactions) are not required to generate the QM-parameterized force field model or use the QM-parameterized force field molecular simulation methods described herein. In some implementations, the simulation software stack 114 can be configured to determine the atomic/molecular polarization at each configuration (e.g., timestep) to support various polarization descriptions in the QM-parameterized force field model. For example, the polarization description can be induced dipole moments. In some implementations, the simulation software stack 114 can include code, when executed, to enable the multipolar Ewald truncation to describe diffuse charge distributions. In some implementations, the simulation software stack 114 can be configured to implement the QM-parameterized force field model which can, in some instances, have an explicit smeared density in addition to multipole moments. The simulation analysis stack 116 can enable analysis, visualization and computation of ensemble properties from atomic and molecular trajectories calculated using the QM-parameterized force field models. In some implementations, the simulation analysis stack 116 can be configured to receive data of locations and energies of atoms at each timestep and generate predictions of ensemble properties (e.g., physical properties) of atoms/molecules.

In some implementations, the parameterization software stack 113 can be configured to receive computed QM data and generate model parameters that can enable the computation of energies and/or forces of a molecular ensemble. The parameterization software stack 113 can include features such as full parameterization, lightweight parameterization, environmental parameterization, QM calculation quality, and/or the like. In some implementations, the full parametrization feature can be applied when the atom is sufficiently different from other atoms of the same chemistry (e.g., aromatic carbon vs. carbon in alkane) such that a full parameterization of the model parameters can be determined. In some implementations, the model parameters can be fitted to, for example, homo and hetero multimer (e.g., dimer) QM energies, monomer properties (ES potential map, conformational energies), multimer QM energies, and/or the like. In some implementations, the lightweight parameterization feature can be applied when it is computationally expensive to obtain QM data. In some situations, the lightweight parameterization feature can be applied only when model parameters may be altered in nature by adjacent atoms, and/or can be fitted largely to monomer properties which are tractable. In some implementations, the environmental parameterization feature can be applied when the atoms and molecules are in a relatively extreme environment. For example, to deduce the liquid and crystal phase parameters from gas phase QM calculations, the transferability can be degraded, when atoms and molecules are in an environment of, for example, high electric fields, in the presence of a nearby ion or a large number of polar groups, in an externally induced field(s), or under high temperature/pressure. For example, a protein can be such an environment. Therefore for such cases the QM parameterized force-field model can be configured to enable an ‘environment’ containing parameterization where the QM energies are computed while the molecules are immersed in a simplified and thermally averaged representation of their environment. The QM calculation quality feature included in the parameterization software stack 113 can balance the QM calculation quality/accuracy with the complexity of the parameterization features of QM-parameterized force field model. In some situations when highly precise predictions of, for example, protein-drug interactions, as well as many other property predictions are desired, a high level of theory (may also be highly computationally expensive) method can be used.

In some implementations, the memory 112 can include and/or store a set of force field features that can be used to optimize and/or improve the QM-parameterized force field model when fitting the model to QM. The set of force field features can be implemented in the simulation software stack 114 and/or the parameterization software stack 113. For example, the set of FF features can include (1) polarizability (e.g., di-polar, quadrupolar, fluctuating charge), and/or exchange-induction (exchange can be ‘polarized’), (2) penetration effects (e.g., charge entities are not points but diffuse multipolar clouds) in electrostatics energies and damping effects in dispersion energies, (3) multipolar electrostatic interaction energy (e.g., with an “interaction rank”, L, of 2 or 3), (4) multipolar exchange, (5) charge transfer interaction, (6) multipolar dispersion interaction, (7) component-based modeling, and/or the like. When constructing the forces and energies of a molecular system, in some implementations, the QM-parameterized force-field model can be configured to decompose the interactions into components with different transferability rules (e.g., the exchange interaction, the dispersion interaction, the electrostatic interaction, the induction interaction, and charge transfer). For example, dispersion interaction can simultaneously have geometric (or multiplicative) rules for the force constants and arithmetic (additive) rule for the effective widths. Electrostatic combination rules can come directly from Coulomb's law applied to continuous charge distributions. In some implementations, the QM-parameterized force-field model can be configured to not force all the energies into transferrable components. In some implementations, there are non-negligible contributions that do not decompose or transfer and the QM-parameterized force-field model can be configured to keep track of these contributions and of the need to determine them later if necessary (while the rest is available via transferability).

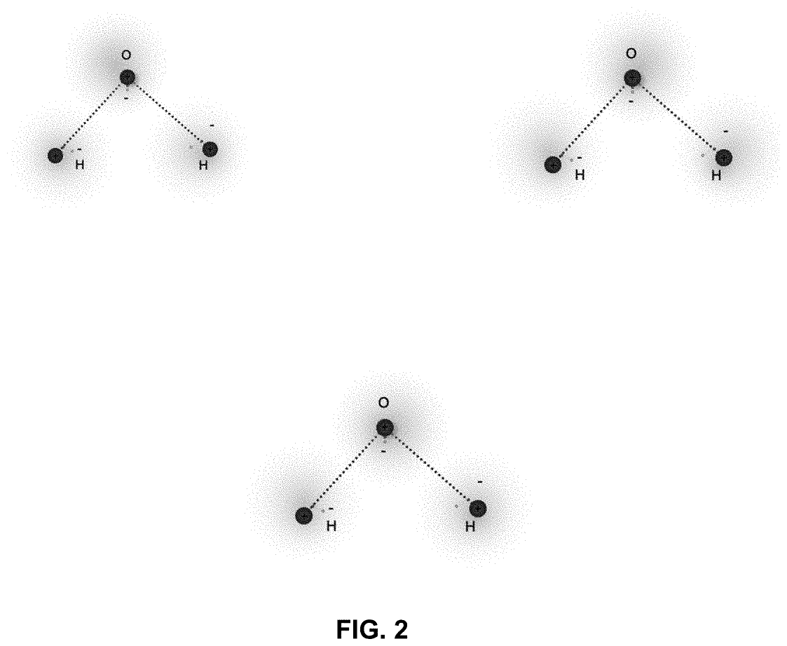

FIG. 2 shows the density-inspired description of interatomic and intermolecular energies and forces, as well as the shift of electron density under external forces (polarization), according to some embodiments.

### Example Molecular Simulation Processes

FIG. 3 shows a flow chart illustrating an example QM-parameterized force-field molecular simulation process 300, according to some embodiments. The example QM-parameterized force-field molecular simulation process 300 can be executed by a processor (e.g., processor 111 in FIG. 1) of a QM-parameterized force-field computing device (e.g., 101 in FIG. 1).

In some embodiments, the example QM-parameterized force-field molecular simulation process 300 can include parameterization of a functional group in a molecular system. Specifically, in some implementations, the parameterization of the functional group includes preparing QM data of molecules containing the functional group of interest and optimizing and/or improving parameters of the QM-parameterized force-field model that describes intermolecular and intramolecular interactions.

At step 301, the example QM-parameterized force-field molecular simulation process 300 includes selecting a molecule/functional group of interest and using, for example, the typification software stack (115 in FIG. 1) to determine an atom type from a set of atom types of each atom in the molecule/functional group of the molecular system. The molecular system includes at least one of homogeneous liquids, a heterogeneous mixture, liquid crystals, or a ligand-protein system. In some implementations, the selected functional group of interest can include at least one of carbon (C), oxygen (O), nitrogen (N), hydrogen (H), phosphorus (P), sulphur (S), chlorine (Cl), fluorine, bromine, and/or iodine. In some implementations, the selected functional group of interest can include at least one of an aromatic carbon, an alkane carbon, an amide nitrogen, a hydroxyl oxygen, a methyl carbon, a carbonyl carbon, a carboxyl carbon, an amino nitrogen, a phosphate phosphorus, or a sulfhydryl sulphur. For example, the functional group of interest can be the —OH alcohol group present in amino acids (e.g., Threonine or Serine), and also present in aliphatic alcohol or drug-like molecules. Such a molecule or a functional group can be referred to as a “molecular target of optimization”. In some implementations, any element from the periodic table or its ionic species can be selected as the functional group of interest. In some implementations, the functional group of interest can be selected by separating the molecule along a rotatable bond or an aromatic segment.

Determining the atom type can be based on, for example, (1) a chemical identity of the atom determined by a number of bound electrons of the atom, (2) properties of a molecular neighborhood of the atom including chemical identities, arrangement and hybridization of a set of atoms neighboring the atom and atom types of each atom from the set of atoms, (c) at least one characteristic of the atom including shape or density of an electronic structure of the atom, effective dispersion coefficients, polarization coefficients, a location of the atom relative to the set of atoms neighboring the atom, and/or (4) the atom's QM monomer properties. In some implementations, the shape and the density of the electronic structure of the atom can include at least one of a dipole moment, a quadrupole moment, or a multipole moment.

At step 302, the example QM-parameterized force-field molecular simulation process 300 includes determining a first set of ab initio molecular properties of a monomer and a second plurality of ab initio molecular properties of a multimer (e.g., dimer) for bonded and non-bonded interactions of the molecule/functional group of interest, based on the plurality of atom types determined from step 301. In some implementations, the QM calculations (e.g., calculations based on quantum mechanics rule-based reasoning) can include determining electrostatic energies on monomers associated with the molecule/functional group (i.e., a first set of ab initio molecular properties of the monomer having the molecule/functional group) and/or multimers (i.e., a second set of ab initio molecular properties of the multimer having the molecule/functional group). The monomer QM calculations (i.e., a first set of ab initio molecular properties of the monomer) can include calculating, for example, molecular moment (e.g., dipole moment, quadrupole moment, multipole moment, at, for example, the mp2/avqz level), molecular electrostatic field or potential, energy of molecular conformation, and polarizability of the monomers or polarization of the monomers by an applied external field or charge. The multimer QM calculations can include calculating, for example, (1) the energies and their components based on a pre-determined standard (e.g., a gold standard, a silver standard, a bronze standard), (2) potential energies of hetero-multimer interactions between the monomer and a first probe species, (3) potential energy of homo-multimer interactions between two identical molecules of the monomer, and/or (4) potential energy of multimer-multimer interactions between the monomer and at least two probe species having a second probe species and a third probe species. The probe species (e.g., the first probe species, the second probe species and the third probe species) can be a charge, a noble gas atom, or a small molecule. In some implementations, the small molecule includes at least one of a water molecule, a methane molecule, or an ammonia molecule. The potential energy of the hetero-multimer interactions, the potential anergy of homo-multimer interactions, and the potential energy of multimer-multimer interactions are calculated based on a set of orientations (or a set of distances) of the hetero-multimer interactions, the homo-multimer interactions, and the multimer-multimer interactions, respectively.

At step 303, the example QM-parameterized force-field molecular simulation process 300 includes determining and optimizing and/or improving multiple parameters of a (polarizable or QM-parameterized) force field model by fitting the QM-parameterized force field model to the monomer QM calculations (i.e., the first set of ab initio molecular properties of the monomer) and/or the multimer QM calculations (i.e., the second set of ab initio molecular properties of the multimer) from step 302 until an agreement between (1) multiple predicted molecular properties and (2) the monomer QM calculations and the multimer QM calculations reaches a threshold. In some implementations, the agreement can be at least one of (1) a first difference between a first set of predicted molecular properties of the monomer and the first set of ab initio molecular properties of the monomer or (2) a second difference between a second set of predicted molecular properties of the multimer and the second set of ab initio molecular properties of the multimer. Fitting the QM-parameterized force field model to the monomer QM calculations and/or multimer QM calculations includes adjusting the values of the parameters of the QM-parameterized force field model to determine and/or predict molecular properties of the monomer and/or multimer so that the molecular properties are in vicinity of the monomer QM calculations (i.e., the first set of ab initio molecular properties of the monomer) and/or the multimer QM calculations (i.e., the second set of ab initio molecular properties of the multimer). For example, fitting the QM-parametrized force field model to the first set of ab initio molecular properties and the second set of ab initio molecular properties includes adjusting at least one parameter from the multiple parameters of the QM-parametrized force field model to cause molecular properties (e.g., electrostatic potential of the monomer) determined using the force field model to be in a pre-determined range of the molecular properties (e.g., electrostatic potential of the monomer) from the first set of ab initio molecular properties or the second set of ab initio molecular properties. For another example, fitting the QM-parameterized force field model to the first set of ab initio molecular properties and the second set of ab initio molecular properties includes adjusting a polarization parameter from the multiple parameters of the QM-parameterized force field model or an induction parameter from the multiple parameters to produce an effect of a probe charge or an external electric field placed around the monomer (or produce a non-additive energy of a multimer), or cause an induction component of dimerization energy of the multimer determined using the QM-parameterized force field model to be in a pre-determined range of the induction component of the multimer from the second set of ab initio molecular properties.

In some implementations, the QM-parameterized force field model can be transferable to a different molecular system from the molecular system such that molecular properties of the different molecular system predicted based on the force field model is within a chemical accuracy of the molecular properties of the different molecular system determined from experiment.

In some implementations, the QM-parameterized force field model includes a plurality of components associated with at least one of exchange energy, dispersion energy, electrostatics energy, charge transfer interaction, induction energy, exchange-induction energy, penetration effect in electrostatics potential, damping effect in dispersion energy, multipolar electrostatics energy, multipolar exchange energy, multipolar dispersion interaction energy, or many-body interaction energy.

In some implementations, the QM-parameterized force field model is associated with intermolecular interactions of the molecular system including an electric field, and/or auxiliary interaction locations including an electron lone pair site or mid-bond site.

At step 304, the example QM-parameterized force-field molecular simulation process 300 includes determining (or predicting), based on the multiple parameters of the QM-parameterized (and/or polarizable) force field model and the QM-parameterized force field model (which can be considered as a quantum mechanics rule-based reasoning technique), molecular properties of the molecule (or the molecular system) within a chemical accuracy (e.g., thermal noise at room temperature and pressure, substantially being 0.59 kcal/mol or kT at 300.) The molecular properties can be ensemble properties (e.g., Boltzmann ensemble properties) predicted using a thermodynamic sampling of the molecular system based on the QM-parameterized force field model. The thermodynamic sampling method can be, for example but not limited to, molecular dynamics simulation, stochastic simulation, or Monte-Carlo simulation, or path integral molecular dynamics (PIMD). In some implementations, the determining the molecular property of the molecular system is based on thermodynamic sampling and nuclear quantum effects (NQE). In some samples, the predicted molecular properties can be ensemble or non-ensemble properties. In some implementations, the predicted molecular property includes at least one of density, heat of vaporization, heat of melting, heat of sublimation, free energy of solvation, or free energy of ligand binding in ligand-protein systems, thermal conductivity, diffusion coefficient, X-ray diffraction, glass transition temperature, glass transition enthalpy change, or enthalpy of solid-solid phase transitions.

At step 305, the example QM-parameterized force-field molecular simulation process 300 can include sending a signal to present the molecular properties of the molecule via, for example, user interface on a monitor of a user's compute device, for visualization and analysis. For example, the signal can include information related to the protein-protein interactions, protein-ligand interactions, protein folding and various protein properties. In some implementations, the signal example QM-parameterized force-field molecular simulation process 300 can be used to screen drug candidate options, assess ligand and protein targets in a lead optimization phase of drug development, in drug design and discovery (e.g., design ligands that bind to a protein for treatment of a disease), in applications of aggregation of biotherapeutic formulations, to predict structure of antibodies, protein-protein binding, among many other applications discussed below. These applications can be performed with the use of the example QM-parameterized force-field molecular simulation process and in response to the signal.

FIGS. 4A-4B show flow charts illustrating an example QM-parameterized force-field molecular simulation process 400, according to some embodiments. Similar to the process discussed with regards to FIG. 3, the example QM-parameterized force-field molecular simulation process 400 can be executed by a processor (e.g., processor 111 in FIG. 1) of a QM-parameterized force-field computing device (e.g., 101 in FIG. 1). In some implementations, an example QM-parameterized force-field molecular simulation process can combine one or more steps of the process 300 in FIG. 3 with one or more steps of the process 400 in FIGS. 4A-4B. In some implementations, one or more steps of the process 300 in FIG. 3 can be replaced with one or more steps of the process 400 in FIGS. 4A-4B to perform a QM-parameterized force-field molecular simulation process.

At step 401, the example QM-parameterized force-field molecular simulation process 400 includes performing a typification process. Specifically, the example QM-parameterized force-field molecular simulation process 400 includes obtaining geometrical coordinates of solute of interest (or molecular target of interest, the functional group of interest) and assigning atom types to the atoms in the molecule.

At step 402, the example QM-parameterized force-field molecular simulation process 400 includes performing QM calculations on the functional group of interest. In some implementations, the example QM-parameterized force-field molecular simulation process 400 can include performing monomer QM calculations (e.g., Molpro) to calculate, for example, moment (e.g., dipole moment, quadrupole moment at, for example, the mp2/avqz level), electrostatic potential, and polarizability of the monomers of the solute of interest (e.g., based on a quantum mechanics rule-based reasoning). The electrostatic potential of the monomers can be calculated for, for example, several Van der Waals radii (one set of radii can be 1.4, 1.6, 1.8, 2.0, 2.5 VdW) and the QM calculated molecular electrostatic potential (MEP) can be fitted at molecular surfaces using an atom-centered point charge model (e.g., as in the Restrained ElectroStatic Potential (RESP) procedure). In some implementations, the monomer QM calculations can include calculating interactions of the molecular target of interest with a number of point charges (the number can be 100 to 200, 200 to 300, 300 to 400, more than 400) and the charge can be 0.1 e (or any suitable charge, e.g., charges <=plus/minus 1e) randomly placed around the molecule.

In some implementations, the example QM-parameterized force-field molecular simulation process 400 can then perform multimer (e.g., dimer) QM calculations based on a pre-determined standard (e.g., a gold standard, a silver standard, a bronze standard). In some implementations, for example, the example QM-parameterized force-field molecular simulation process 400 can perform the multimer QM calculations based on the silver standard, defined as:

E_{silver_standard}=mp2/CBS+ccsd(t)/aug-cc-pVDZ-mp2/aug-cc-pVDZ

where mp2/CBS can be obtained by extrapolation using mp2/aug-cc-pVTZ and mp2/aug-cc-pVQZ intermolecular energies. Example multimers can include: the solute of interest and water, the solute of interest and methane, the solute of interest and the solute of interest (i.e., homo-multimers). In some implementations, to perform the multimer QM calculations, the QM-parameterized force-field computing device can extract transferable components from the interaction of the multimers and with sufficient chemical accuracy (e.g., 0.5 kcal/mol or less) for total energies. In some implementations, the QM-parameterized force-field computing device can extract sub-components of the energetic interactions of the multimers. In some implementations, the QM-parameterized force-field computing device can use Symmetry-Adapted Perturbation Theory (SAPT) methods (or other energy decomposition analysis (EDA) methods) to extract such components. A specific example of such a decomposition (i.e., extracting transferable components from the interaction of the multimers, or extracting sub-components of the energetic interactions of the multimers) can be dimerization energy calculated with DFT-SAPT/aug-cc-pVTZ with PBE0 functional asymptotically corrected. A collection of computer codes can be designed to implement the SAPT methods.

In some implementations, the QM-parameterized force-field computing device can calculate the energies and their components at a chemical accuracy no less than the desired accuracy of output predictions. For example, if the desired input accuracy is on the average 0.1 kcal/mol, a quantum level of theory can be mp2/CBS+ccsd(t)/aug-cc-pVDZ-mp2/aug-cc-pVDZ. For another example, if the desired level of input accuracy is 0.01, the quantum level of theory can be mp2/CBS+ccsd(t)/aug-cc-pVTZ-mp2/aug-cc-pVTZ. If the desired level of input accuracy is 0.6 kcal/mol the quantum level of theory can be DFT-SAPT aug-cc-pVTZ with the PBE0 functional asymptotically corrected or mp2/aTZ. If the desired level of accuracy is 1.5 kcal/mol the quantum level of theory is HF (Hartree-Fock) 6-31G*.

In some implementations, the accuracy mismatch between QM methods suitable for extraction of full energy and QM methods suitable for extraction of components can be resolved by placing the mismatch into a suitable component, for example, electrostatic, dispersion, exchange, charge transfer or polarization components.

In some implementations, to generate the multimer energy input data, the QM-parameterized force field computing device can calculate a set of multimer interactions of “molecular target of optimization” (e.g., ethanol) with probe molecules. Probe molecules can include water, methane, ammonia, ions, or any preferably small molecular entities. The set of multimer interactions can include, for example, a set of surrounding neighborhoods of the molecule of interest with varying intermolecular distance placed in random orientations, or a set of potential minima and saddle points of intermolecular potential energy surface which can be generated via a random search, a directed search, a simulated annealing procedure, a stochastic optimization procedure, a genetic optimization algorithm, and/or the like. The set of multimer interactions can also include, for example, an examination set of simple translations and rotations around major minima, which can be used as input data and as a visual examination check of the interaction and its components. The set of multimer interactions can include, for example, other inter-molecular configurations that help define the potential energy surface and/or configurations generated to represent more specifically the interactions of interest in a system. Such configurations can be, for example, an ion and neighboring amino acid residues encountered in proteins, adjacent molecules in a crystal lattice, a chemical entity of special importance (e.g., a solvent).

At step 403, the example QM-parameterized force-field molecular simulation process 400 can receive and process the QM data (e.g., the monomer QM calculations and the multimer calculations obtained in step 402, the geometrical coordinates obtained in step 401) and output the results to, for example, the parameterization software stack 113 (e.g., MolMechFitter) for optimization and/or improvement of the parameters of the QM-parameterized force field model. In some implementations, the processing of the received QM data includes generating an initial prediction of the parameters of the QM-parameterized force field model based on, for example, analogous atom-type parameters existing in the repository (e.g., FF models 117 in memory 112 in FIG. 1, or a database). In situations where no existing parameters can be found, the QM-parameterized force-field computing device can generate non-zero numbers as the initial values of the parameters.

At step 404, the example QM-parameterized force-field molecular simulation process 400 includes determining and optimizing the parameters of the QM-parameterized force-field model using the parameterization software stack (e.g., 113 in FIG. 1). In some implementations, the QM-parameterized force field computing device can perform the optimization and/or improvement of atomic and molecular interaction parameters. Such optimization and/or improvement can, for example, be performed either in toto with the input data (e.g., the monomer QM calculations and the multimer calculations obtained in step 402, geometrical coordinates obtained in step 401, initial parameters of the QM-parameterized force field model), or in steps of:
1) Determine, based on monomer property calculations, parameters that correspond to electrostatic multipoles and polarizabilities on atoms inside the functional group (e.g., OH and connections to such OH). In this step charge shift parameters can be fitted as well as polarizabilities and their anisotropy to the properties described in monomer QM calculations. This can produce an initial prediction for such parameters.
2) Optimize and/or improve dispersion parameters which can be fitted to the dispersion component of QM calculation that are described in multimer QM calculations.
3) Optimize and/or improve electrostatic and exchange-repulsion components of the full energy of the interaction of the functional group of interest (or the molecule). In some implementations, all multipole electrostatic related charge shifts can be optimized and/or improved starting from parameters obtained in the initial prediction of such parameters from step 1) above as well as electrostatic and exchange-repulsion related widths and force constants.
4) Achieve agreement for the total energies of multimers by optimizing and/or improving polarization related parameters (starting from the initial prediction of such parameters from step 1) above) as well as pair-specific constants that can correspond to, for example, charge-transfer interactions.
5) Optionally perform final refinement of the parameters that describe intermolecular interactions.

These steps of optimization and/or improvement can be performed in different orders and in some implementations, not all steps are required at all time. Some steps of the optimization and/or improvement can be optional.

At step 405, the QM-parameterized force field computing device can check whether the total or component energy differences of multimers (FF−QM) are within a pre-determined chemical accuracy. If pre-determined chemical accuracy has not been achieved, the QM-parameterized force field computing device can repeat all or some steps in step 404 to optimize and/or further improve the parameters (or introduce new parameters). In some implementations, the QM-parameterized force field computing device can perform other solutions to optimize and/or improve the parameters (e.g., extend current atom typification, alter the weights on the total energy or energy components to improve the agreement between FF and QM).

If the pre-determined chemical accuracy has been achieved, the QM-parameterized force field computing device can proceed to step 406. At step 406, the example QM-parameterized force-field molecular simulation process 400 includes molecular dynamics simulations. In some implementations, the example QM-parameterized force-field molecular simulation process 400 can include inputting the parameters of the QM-parameterized force field model determined from steps 404 and 405 into the simulation software stack (114 in FIG. 1). In some implementations, the example QM-parameterized force-field molecular simulation process 400 can prepare solute in solvent (water or cyclohexane) box grid (via the simulation software stack 114) and run molecular dynamics simulations based on, for example, Classical and Path-Integral methods using proper variations of solute interaction with solvent in alchemical lambda space. In some implementations, an ambient temperature and pressure of T=298 K and P=1 atm (using appropriate thermostat/barostat) can be provided into the simulation software stack 114 as the surrounding environment.

At step 407, the example QM-parameterized force-field molecular simulation process 400 includes analysis of the molecular dynamics simulations using, for example, the simulation analysis stack (116 in FIG. 1). For example, the example QM-parameterized force-field molecular simulation process 400 can use Bennett Acceptance Ratio (BAR) to compute the free energy of hydration/solvation of the molecule in water or any other solvent. In some implementations, the analysis of the molecular dynamics simulations includes analysis, predication, and visualization of ensemble properties of atoms/molecules from atomic and molecular trajectories using the optimized parameters of the QM-parameterized force field model (i.e., the quantum mechanics rule-based reasoning technique) and the QM-parameterized force field model.

FIG. 4B shows example detailed sub-steps of the parameterization and optimization and/or improvement of the parameters of the QM-parameterized force field model in step 404 of FIG. 4A, in some embodiments. In some embodiments, the example QM-parameterized force-field molecular simulation process 400 can include inputting data into the parameterization software stack (e.g., 113 in FIG. 1) The input data can include QM dispersion energy input 411 (e.g., Complete Basis Set (CBS) extrapolated solute-probe hetero and homo (methane, water, self) multimer dispersion energies from Total energy using silver standard) and the initial guess for force field parameter values 412 (e.g., values taken from analogous atoms or initial non-zero numbers). At step 413, the example QM-parameterized force-field molecular simulation process 400 can optimize and/or improve, based on the input data, dispersion parameters (w_{DS},C₆,C₈) of the force field model. At step 415, the example QM-parameterized force-field molecular simulation process 400 can optimize and/or improve charge shift parameters, based on the dispersion parameters and QM calculations of monomers 414 (e.g., QM electrostatic input including monomer based ESP map+dipole, quadrupole moments of the solute computed at MP2/atz level).

At step 416, the example QM-parameterized force-field molecular simulation process 400 can further refine the charge shift parameters (from step 415) and optimize and/or improve the electrostatic and exchange widths for the force field model, based on QM calculations of multimers 417 (e.g., electrostatic and exchange energy of multimers E1pol & E1exchange of solute-probe multimer energy DFT-SAPT/avtz.) At step 419, the example QM-parameterized force-field molecular simulation process 400 can optimize and/or improve the induction parameters and charge transfer and dispersion corrections, based on QM calculations of total energy of homo and hetero-multimers 418 (e.g., total multimer interaction energy calculated at silver standard.) At step 420, the example QM-parameterized force-field molecular simulation process 400 can further refine any or all force field parameters based on steps 415, 416, 419 and QM calculations of total energy of homo and hetero-multimers 418. The example QM-parameterized force-field molecular simulation process 400 can then proceed to step 405 from FIG. 4A to check whether the total or component energy differences of multimers (FF−QM) are within a pre-determined chemical accuracy.

### Example

The example here illustrates using the QM-parameterized force field model and the QM-parameterized force field molecular simulation described with regards to FIG. 1-FIG. 4B to determine properties of a biophysical molecular system (specifically applying to a biophysical molecular system relating to solvation of solutes in the solvents). The liquid state properties of a biophysical molecular system can play a key role in, for example, battery design, and are a major part of more structured biological ensembles: e.g., protein shape and behavior, protein-ligand complexes and cell membranes, solid state properties of ceramics, and/or the like.

For biophysical ensembles that exist at room temperature and pressure, and where the entropic contributions are on par with interaction strengths, the free energies can be both important and difficult to predict. In this example, free energies of solvation are computed for a diverse set of organic compounds using a (polarizable) QM-parameterized force field model fitted, in some implementations, entirely to ab initio calculations (without fitting to experimental data). The mean absolute errors of hydration, cyclohexane solvation, and corresponding partition coefficients can substantially reach 0.2 kcal/mol, 0.3 kcal/mol and 0.22 log units, i.e. within chemical accuracy.

The predictive ability of the QM-parameterized force-field model described herein can be demonstrated by computing solvation free energies for a wide range of chemical functional groups in, for example, water and cyclohexane. The QM-parameterized force-field molecular simulation method described in this example can be executed by the processor of the QM-parameterized force-field computing device (e.g., 101 in FIG. 1).

### Abbreviations

SI: Supplementary Information

QM: Quantum Mechanics

FF: force-field (model)

MAE: Mean Absolute Error

NQE: Nuclear Quantum Effect

MD: Molecular Dynamics

PIMD: Path Integral Molecular Dynamics

H2O: Water

CHEX: CycloHexane

MMFF94: Merck Molecular Force Field

AMOEBA: Atomic Multipole Optimized Energetics for Biomolecular Simulation

GAFF: Generalized AMBER (Assisted Model Building with Energy Refinement) Force Field

A. QM and Force Field agreement (“QM-FF agreement”)

An advantage of the QM-parameterized force-field model described herein includes determining the degree of faithfulness that is sufficient for modeling the liquid phase of arbitrary molecules and mixtures while keeping the model complexity manageable. The QM-parameterized force-field model and the QM-parameterized force field molecular simulation method described herein provide an ab-initio-based molecular interaction toolset with chemical accuracy that can enable the long-promised quantitative in-silico determination of properties of any molecular ensembles.

In this example, the first step is choosing the level and accuracy of the underlying QM computations. The chosen accuracy level of input data can propagate throughout the technology stack and can affect the accuracy of output predictions. The QM-parameterized force-field computing device can fit the intermolecular interactions to multimer and select multimer QM energies at, for example, the highest level of theory practical for large-scale parameterization. This specific level can have an average accuracy of 0.05 kcal/mol on a diverse set of small molecular configurations as compared with the most precise and computationally demanding methods. (This is referred to as the “silver standard”.)

The QM-parameterized force-field computing device can then encapsulate the QM interaction energies in a physics-based analytical model. The faithfulness can introduce a significant level of complexity from the functional form: polarizability terms enable proper transferability from multimer to bulk energies; multipole descriptions of both the electrostatic and exchange-repulsion interactions permit a precise fit of the potential energy surface for multimer orientations; a fairly detailed typification accounts for the difference in interaction properties of identical atoms in diverse chemical environments.

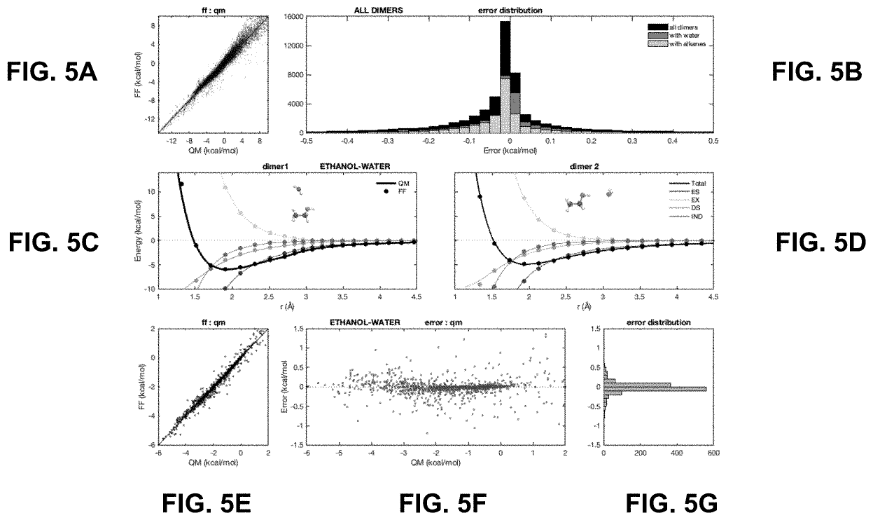

FIGS. 5A-5G illustrates the correspondences and deviations between the QM calculations and force field model predictions, in some embodiments. The deviation (also calculated as “errors”) between QM and FF energies can be referred to as the QM-FF agreement, The QM-parameterized force-field molecular simulation process (e.g., 300 in FIGS. 3 and/or 400 in FIGS. 4A-4B) includes determining whether the agreement between (1) multiple predicted molecular properties and (2) the monomer QM calculations and the dimer QM calculations has reached a threshold as discussed in step 303 with regards to FIG. 3, and/or the step 405 with regards to FIG. 4A. As shown in FIGS. 5A and 5B, the deviation (MAE) between QM and FF energies for the benchmark dimers and multimers in the training set is around 0.17 kcal/mol and the error distribution is centered around zero. The functional form reproduces the lower energy conformations very well and, in some implementations, is designed to permit a larger error in less important high-energy high electron overlap regions. Error distribution in FIG. 5B shows the distribution of errors for the training dimer sets. In some calculations and simulations, the MAE of errors can be around 0.17 kcal/mol for all dimers in the training set, 0.19 kcal/mol for dimers with water (total number of dimers=36309), and 0.16 kcal/mol for dimers with alkanes (total number of dimers=25986).

FIGS. 5C-5G illustrate the QM-FF agreement for a specific single representative system, a strongly interacting ethanol-water dimer. FIGS. 5C and 5D show dissociation curves for primary and secondary minima of the ethanol-water dimer, respectively. QM energies are represented as lines and FF values are represented as filled circles in FIGS. 5C and 5D. The different line styles designate the energy components: electrostatics (ES), exchange-repulsion (EX), dispersion (DS) and induction (IND). The QM-FF agreements for the total energy and for each component meet a pre-determined threshold, a chemical accuracy within 0.1 kcal/mol. FIGS. 5E-5G show error distributions for the ethanol-water dimer. The QM-FF agreement for the ethanol water dimer system, as shown in FIG. 5E, is similar to the QM-FF agreement shown in FIG. 5A. The error (y-axis of the plot) versus QM (x-axis of the plot) calculation, as shown in FIG. 5F, provides a difference plot offering a more detailed view and is projected onto error distribution. The MAE for the errors in this system is around 0.08 kcal/mol, as shown in FIG. 5G.

### B. Simulations

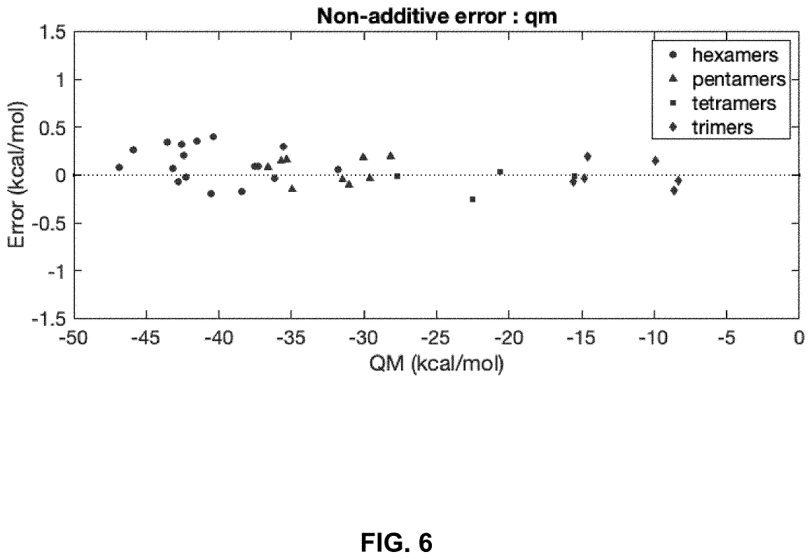

In some simulations, the example QM-parameterized force-field molecular simulation process includes using the functional form of the bonded interactions, with force constants and equilibrium values fitted to QM energies at the MP2/aug-cc-pVTZ level of theory. In some simulations, solvents of water and cyclohexane are selected. Cyclohexane (CHEX) is nonpolar and equilibrates relatively quickly. Reliable experimental data for both CHEX solvation free energies, as well as for the CHEX/H2O partition coefficients are used in these simulations. In some instances, though the two molecules (water and cyclohexane) were parameterized with the same procedure as every other functional group, the two molecules participate in bulk and can benefit from extra examination of their liquid-state properties (e.g., considering the molecular 2-body interactions and the multi-body contributions.) For water, which is small, polar, and polarizable, the multibody energies are estimated to be a sizable 27% of the total energy. FIG. 6 shows the non-additive many-body error of selected optimized water multimers (e.g., hexamers, pentamers, tetramers, and trimers) in relation to the total QM intermolecular energy. In some simulations, all of the many-body errors are below 0.5 kcal/mol, or below 1% of total energy and below 3% of the many-body contributions, confirming that the energy partitioning and the induction terms of the QM-parametrized force field model described herein capture the non-additive fraction properly.

Biological systems exist mostly at room temperature and pressure, where the shifting interplay between enthalpy and entropy enables the immense variety of biological phenomena. The QM-parameterized force field molecular simulation process described herein enables correctly predicting the free energies of ensembles. For solvation, in addition to capturing the enthalpy of interaction with itself and the solute, a solvent model can reproduce the entropic effects of pushing aside and reordering molecules to create a cavity for placing the solute. Water molecules are small, highly polar, and highly structured at room temperature and pressure. Table 1 lists three bulk properties for each of the two solvents (water and CHEX): density, heat of vaporization and the highly informative self-solvation. The predicted values, using the QM-parameterized force field model (labeled as AFF (PEID8) for water and AFF (PIMD) for CHEX), for water agree with experimental values to within 3% or better. The predictions using the QM-parameterized force field model are significantly closer to the experimental values than the classical MD simulations (labeled as AFF (MD) for both water and CHEX). The predicted values of cyclohexane, comparing with the experimental values, present an agreement to a lesser degree than the agreement of water. Given that cyclohexane is a larger molecule, the energetics are meeting the pre-determined threshold. In addition, when predicting the properties of cyclohexane, the QM-parameterized force field molecular simulation includes assigning same atom types to atoms of the cyclohexane (similar to the step 301 in FIG. 3 or step 401 in FIG. 4A). The atom types are assigned as linear alkanes (instead of smaller, strained, cyclic alkanes) in these simulations, which may introduce a slight discrepancy with QM energies. The QM-FF agreement of bulk energetics of cyclohexane are within the chemical accuracy of 0.5 kcal/mol.

TABLE 1

| | Density | Hvap | Hydration | self-solvation |
|---|---|---|---|---|
| H2O | (g/cc) | (kcal/mol) | (kcal/mol) | (kcal/mol) |
| experimental | 0.997 | 10.51 | −6.30 | −6.30 |
| AFF (MD) | 1.027 | 11.98 | −6.81 | −6.81 |
| AFF (PIMD8) | 1.027 | 10.63 | −6.13 | −6.13 |
| | Density | Hvap | Hydration | self-solvation |
| CHEX | (g/cc) | (kcal/mol) | (kcal/mol) | (kcal/mol) |
| experimental | 0.790 | 7.91 | 1.20 | −4.42 |
| AFF (MD) | 0.803 | 8.04 | −0.10 | −4.23 |
| AFF (PIMD) | 0.786 | 7.83 | 1.08 | −4.02 |

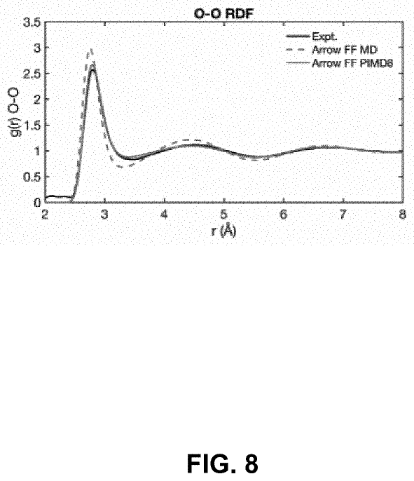

FIG. 8 shows the predictions of radial distribution function (RDF) of oxygen-oxygen (O—O) distance in water, using the QM-parameterized force field model, in some embodiments. The predictions using the QM-parameterized force field model (labeled as Arrow FF PIMD8) are significantly closer to the experimental values (bolded solid lines) than the classical MD simulations (dotted lines labeled as Arrow FF (MD) for the RDF for the O—O distance in water).

### Solutes and Solvation Predictions

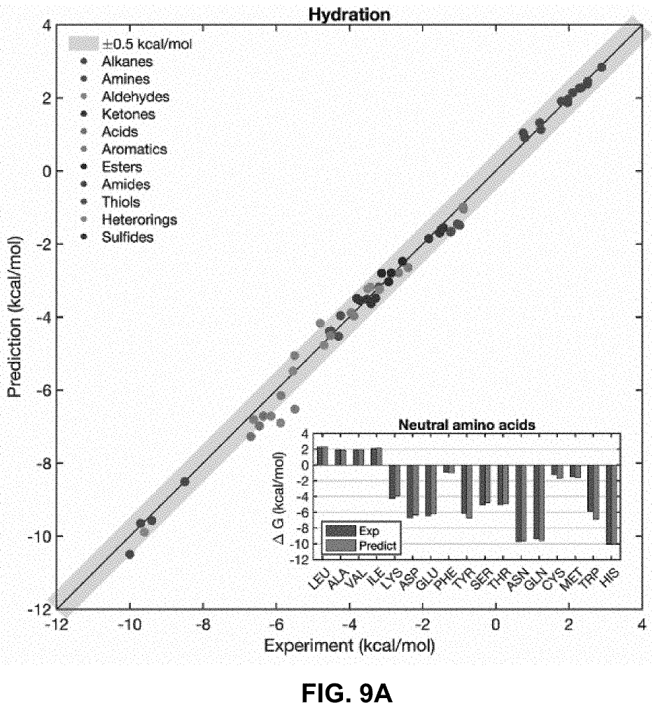

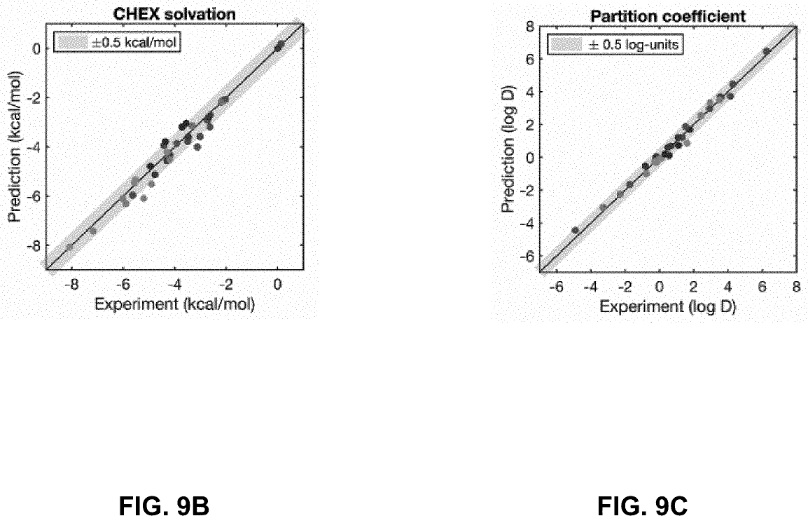

In some example simulations, solutes containing neutral chemical functional groups, for example carboxylic acids, alkanes, alkenes, aromatics, aldehydes, ketones, alcohols, amides, esters, thiols, sulfides, disulfides and heterocycles, are used. The simulations were performed independently by four groups using their own respective computational resources and architectures, and then averaged. FIGS. 9A-9C show solvation and hydration free energies predictions of various solutes, using the QM-parameterized force field model, in some embodiments. The insert figure in FIG. 9A shows the predictions of neutral amino-acid, using the QM-parameterized force field model, because of the importance of aqueous protein and protein-ligand systems and accurate predictions of solvation and desolvation of amino acids. FIG. 9C shows the partition coefficient for the cyclohexane simulations (as shown in FIG. 9B) which can be a measure of the model's simultaneous compatibility with both polar (e.g., aqueous) and non-polar (e.g., membranes and proteins) environments.

Specifically, FIG. 9A shows the predicted vs. experimental free energy of hydration for a diverse set of compounds. The straight line is a line of agreement between experimental and simulated/predicted values, and the gray bar is the range or chemical accuracy. FIG. 9B shows the predicted vs. experimental free energy of solvation in cyclohexane. The molecules here are a subset of those in a) because only those with experimental values for CHEX solvation can be included. In some simulations/predictions, the error (MAE) for the free energies of hydration is 0.2 kcal/mol and for the neutral amino-acid subset is 0.23 kcal/mol. Some hydration errors for o-cresol and 3-methyl-indole can be only −1 kcal/mol. For solvation in cyclohexane the MAE is 0.3 kcal/mol, and for the partition coefficient it is 0.22 log units. These predictions uniformly meet the pre-determined chemical accuracy across a diverse range of chemical groups of varying sizes and interaction strengths.

In some implementations, the QM-parameterized force field model takes into consideration or includes parameters relating to, for example, accurate treatment of long range electrostatic (PME) (and dispersion interactions, proper thermodynamic modelling (thermostats and barostats), enhanced sampling techniques and the Path Integral formulation of nuclear motion. In some simulations, NQE are taken into consideration in the QM-parameterized force field model for accurate predictions for molecular systems.

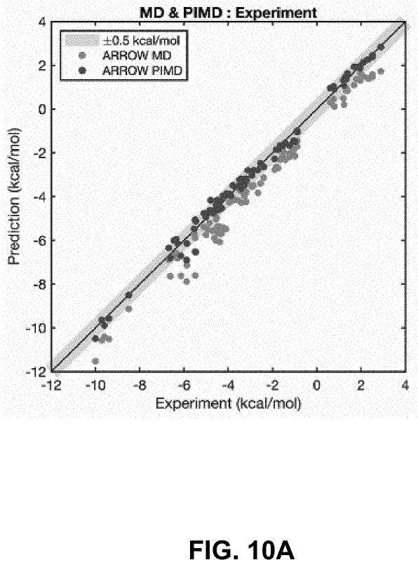

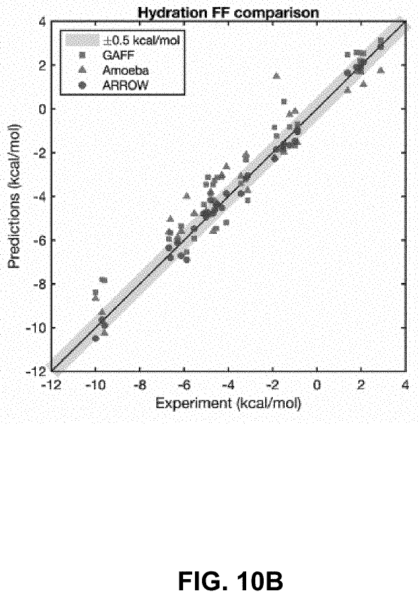

FIGS. 10A-10B show predicted and experimental results of the hydration including predicted results using the QM-parameterized force field model, in some embodiments. The predicted results using the QM-parameterized force field model (e.g., including NQE) systematically improves the predictions (e.g., compared with classical MD values) and yields a greater agreement with the experimental values. The overall error (MAE) is reduced from 0.78 to 0.2 kcal/mol.

FIG. 10B shows a comparison of the hydration free energies using the QM-parameterized force field model with known wide coverage force field models. The errors (MAE) for General Amber Force Field (GAFF, a representative of fixed-charge models), a polarizable model AMOEBA and the QM-parameterized force field model (ARROW FF) are 0.88, 0.76 and 0.22 kcal/mol respectively.

Table 2 shows time required for computation of 1 ns molecular dynamics solvation simulation for each window using the QM-parameterized force field model based on classical and path-integral methods. In some implementations, these timings do not employ the various speed-up techniques that may result in energy errors >0.1 kcal/mol. For example, in these implementations, centroid RPMID was not used.

TABLE 2

| GPU/CPU type | MD | PIMD 8 | |
|---|---|---|---|
| GeForce RTX 2080 | ~2 hours | ~16-18 | hours |
| TI + 2 CPU cores | | | |
| CPU 8 cores | ~10 hours | ~78 | hours |

### C. Methods

In some simulations, the total dimer energies are calculated with silver standard i.e. MP2/CBS, calculated with Helgaker cubic extrapolation from aug-cc-pVTZ->aug-cc-pVQZ+CCSD(T)/aug-cc-pVDZ-MP2/aug-cc-pVDZ. To improve transferability and ease optimization, in some implementations, DFT-SAPT decomposition is used with dHF correction at aug-cc-pVTZ level with PBE0 functional asymptotically corrected4. In these implementations, four parts of DFT-SAPT decomposition are implemented in the QM-parameterized force field model. ES (electrostatic E1pol), EX (exchange-repulsion Elexch), IND (induction, E2ind+E2ind-exch+δHF), DS (dispersion, E2disp+E2disp-exch+Esilver-standard−DFT-SAPTtotal_energy). The dispersion term can accumulate all disagreements between total energy described by the “silver standard” and DFT-SAPT+SHF energies.

In some implementations, the QM-parameterized force field model (also referred to as ARROW-FF) includes features: the two-body non-bonded interactions are composed of electrostatic, exchange-repulsion and dispersion terms, and many others. The electrostatic and exchange-repulsion terms are multipolar, and their radial dependence is a Slater-like exponential to describe charge penetration and delocalization. The dispersion term can be represented by a spherical, 2 term (C6 and C8), Tang-Toennies-damped interaction. Multibody effects can be modeled by anisotropic atomic polarizable dipoles interacting with the electrostatic term and with each other and iterated to self-consistent field (SCF) convergence on every non-bonded step. In some implementations, there are no explicit three-body terms or charge flux. The intermolecular parameters of the QM-parameterized force field model are determined by agreements with QM values of dimer and multimer energies, electrostatic potentials, and multipole moments of monomers. To aid transferability, the QM-parameterized force field molecular simulation process can include matching the individual components to their corresponding QM counterparts, in addition to reproducing the total energy. The bonded interactions are fitted to monomer QM energies computed at the df-MP2/aug-cc-pVTZ level of theory.

In some simulations of the QM-parameterized force field molecular simulation process, the molecules were placed in a water or cyclohexane box of size 32×32×32 Å3. Particle-Mesh Ewald (PME) algorithm was used to compute the electrostatic interactions. A 32×32×32 grid mesh and 5th power spline interpolation order can be used to compute the inverse PME sum. 9 Å cutoffs were used to compute the direct PME sum as well as the exchange and dispersion interactions. Bulk corrections were applied to account for distance cut-off of dispersion interactions. Solvation free energies were computed by decoupling the interactions between the solute and the solvent molecules. Electrostatic, exchange-repulsion and dispersion interactions were switched off simultaneously using a lambda-dependent scaling (a scaling factor power=2 was used) and a soft-coring algorithm (maximal soft-coring radius=1.5 Å, soft-coring radius scaling factor power=1). 15 lambda points unequally spaced (X=0, 0.05, 0.1, 0.2, 0.3, 0.4, 0.5, 0.6, 0.7, 0.75, 0.8, 0.85, 0.9, 0.95, 1.0) were used to decouple the solute-solvent intermolecular interactions.

In some simulations of the QM-parameterized force field molecular simulation process, energy minimization (10,000 steps) using the steepest descent algorithm was initially performed on all the simulation systems. This was followed by a 50 ps equilibration and 1 ns production runs in the isothermal-isobaric ensemble (NPT). The temperature was maintained at 298 K using a Nose-Hoover thermostat (chain length=6, relaxation time=1 ps). Pressure was maintained at 1 atm using a MTTK barostat (relaxation time=5 ps). A Multiple Time Step (MTS) algorithm was used to integrate the equations of motion both for the classical and path-integral runs. Bonded and PIMD beads interactions were integrated with the time step of 0.125 fs, while intermolecular interactions were integrated with a time step of 2 fs. The calculations were performed using the QM-parameterized force field model.

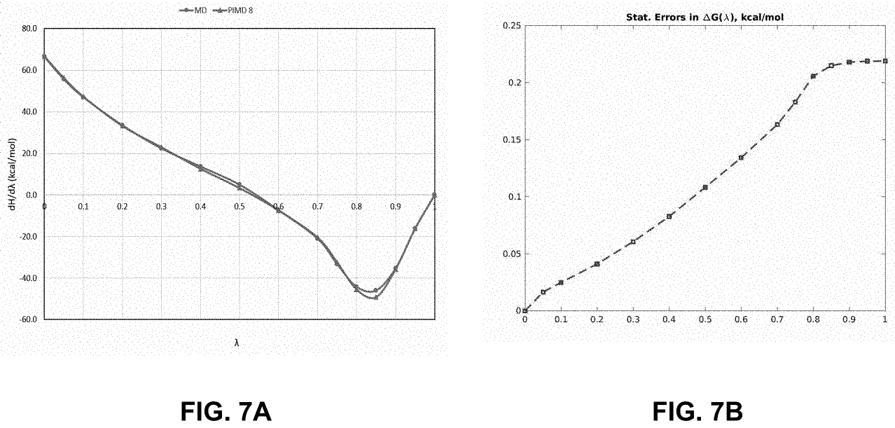

Using the QM-parameterized force field model, the QM-parameterized force field device can compute or predict the solvation free energies using both the Bennett acceptance ratio (BAR) and thermodynamic integration (TI) method with <dH/dλ> values interpolated by cubic splines. FIGS. 7A-7B show a <dH/dλ> dependence for MD and PEID (8 replicas) on desolvation coordinate λ (anthracene in water). For a 1 ns trajectory and for different λ states the estimated statistical error of computed free energy of solvation is about 0.2 kcal/mol (see FIG. 7B). This value is close to the observed RMS errors for computed solvation ΔG vs experiment and to the estimated error of experimental solvation ΔG values. FIG. 7A illustrates a <dH/dλ> dependence for desolvation of Anthracene in water. FIG. 7B illustrates accumulated statistical errors for TI calculations of AG desolvation of Anthracene in water. Additionally, FIGS. 7A and 7B show the agreement between the force field predictions and experiment hydration of anthracene, a large, non-polar molecule, is correct to within 2% (0.1 kcal/mol).

Example embodiments described herein include a QM-parameterized force-field model and a QM-parameterized force field molecular simulation process that can predict free energies of solvation of arbitrary molecules to an accuracy better than thermal noise at room temperature (˜0.5 kcal/mol). The correspondence from quantum mechanics to ensemble predictions meets the chemical accuracy for reasons including, but not limited to, first, the benchmark QM calculations can preserve a degree of accuracy. Second, the QM-parameterized force-field model can provide a description of the QM potential energy surface (PES), which imposes a significant yet computational manageable level of complexity on the functional form. Third, the averaging of molecular ensemble can be performed with care. Finally, the dynamics of sampling of the system can account for nuclear quantum effects.

### Example QM-Parameterized Force Field Model

A) Charge Density

In the QM-parameterized force field model, the charge density of the electron cloud of atom ‘a’ located at position Ra is written as:

$${ρ}_{a} ( r ) = {\hat{D}}_{a} \frac{{q}_{a}}{8 π {w}_{A}^{3}} exp ( - \vert r - {R}_{a} \vert {Iw}_{A} ) , \qquad ( A1 )$$

where qₐ is the cloud charge and w_{A} is the atom-type parameter that characterizes the cloud size. Capital letters ‘A’, ‘B’, . . . to denote the atom-types respectively for atoms ‘a’, ‘b’, . . . ; similar notations are used for bond, bend and torsion atoms types (e.g. ‘AB’ denotes the bond type for the pair of bonded atoms ‘a’ and ‘b’).

The cloud charge is written in the form:

$${q}_{a} = - {Z}_{A} + {\sum }_{b ∈ \{ a \}} {q}_{BA} \qquad ( A2 )$$

where Z_{A} is the charge of the core for the atom type ‘A’, {a} denotes the set of atoms chemically bonded to atom ‘a’ and q_{BA} is the bond charge transfer parameters (thus the equality q_{AB}=−q_{BA} providing for charge conservation).

Differential operator \hat{D}ₐ

$${\hat{D}}_{a} ≡ 1 + {t}_{a} \frac{\partial }{\partial {R}_{a}} + \frac{1}{2} {( \frac{\partial }{\partial {R}_{a}} )}^{T} {ω}_{a} \frac{\partial }{\partial {R}_{a}} \qquad ( A3 )$$

where vector tₐ and 3×3 tensor ωₐ introduce p and d type density anisotropy, respectively. They are related to the local dipole Pₐ and quadrupole Qₐ as:

Pₐ=qₐtₐ,

Qₙ=qₙωₙ (A4)

Vector tₐ is a sum of permanent and induced vectors:

tₐ=tₐ^{per}+tₐ^{ind} (A5)

The induced part is written as

tₐ^{ind}=t_{A}^{max}τₐ (A6)

where t^{max}_{A} is the atom-type parameter and τₐ is the dimensionless vector representing the dynamic variable, its length being confined within interval (0, 1) by the restraint potential U_{IN}.

The permanent part in Eq. (A5) is written as:

$${t}_{a}^{per} = {\sum }_{b ∈ \{ a \}} {t}_{AB} {n}_{ab} \qquad ( A7 )$$

where n_{ab} is the unit vector, n_{ab}=R_{ab}/R_{ab}| and R_{ab} is the vector directed from atom ‘a’ to atom ‘b’ and t_{AB} is the bond-type parameter associated with permanent ‘chemical’ polarization along the bond ‘ab’ (t_{AB}≠t_{BA}).

As for the local quadrupole tensor, its components are calculated as:

$${( {ω}_{a} )}_{αβ} = 2 {\sum }_{b ∈ \{ a \}} {Q}_{AB} ( {( {n}_{ab} )}_{α} {( {n}_{ab} )}_{β} - \frac{{δ}_{αβ}}{3} ) \qquad ( A8 )$$

where α,β=x, y, z and Q_{AB} are the bond-type parameters.

The atom core density (proportional to Dirac's delta-function) can be formally considered as the limiting case of Eq. (A1) replacing qₐ by Z_{A}, dropping operator \hat{D}ₐ and formally approaching zero by the width parameter.

B) Non-Valence Interactions

### Electrostatic Potential

Based on representation of Equation (A1) the potential φ_{ab} of electrostatic interaction of two unit charge distributions ‘a’ and ‘b’ separated by a distance R_{ab}=|R_{b}−Rₐ| can be represented in the form:

$${φ}_{ab} ( {R}_{ab} , {t}_{a} , {t}_{b} ) = {\hat{D}}_{a} {\hat{D}}_{b} {φ}_{ab}^{( 0 )} ( {R}_{ab} ) \qquad ( B1 )$$

$${φ}_{ab}^{( 0 )} ( r ) = \frac{1}{r} [ 1 - f ( {w}_{A} , {w}_{B} ) {e}^{- r / {w}_{A}} - f ( {w}_{B} , {w}_{A} ) {e}^{- r / {w}_{B}} ] \\ f ( u , v ) ≡ \frac{{u}^{4} ( 3 {v}^{2} - {u}^{2} )}{{( {v}^{2} - {u}^{2} )}^{3}} + \frac{{u}^{3}}{2 {( {v}^{2} - {u}^{2} )}^{2}} r . \qquad ( B2 )$$

Expanding equation (B1) one gets:

$${φ}_{ab} ( {R}_{ab} , {t}_{a} , {t}_{b} ) = {\sum }_{k = 0}^{4} {F}_{ab}^{( k )} ( {R}_{ab} , {t}_{a} , {t}_{b} ) {φ}_{ab}^{( k )} ( {R}_{ab} ) \qquad ( B3 )$$

where F's are the algebraic factors defined as:

$${F}_{ab}^{( 0 )} = 1 , \qquad ( B4 )$$

$${F}_{ab}^{( 1 )} = ( {t}_{b} {R}_{ab} ) - ( {t}_{a} {R}_{ab} ) - ( {t}_{a} {t}_{b} ) ,$$

$${F}_{ab}^{( 2 )} = - ( {t}_{a} R ) ( {t}_{b} R ) + \frac{1}{2} Sp {ω}_{a} {ω}_{b} + \frac{1}{2} ( {R}^{+} {ω}_{a} R ) + \frac{1}{2} ( {R}^{+} {ω}_{b} R ) + ( {R}^{+} {ω}_{a} {t}_{b} ) - ( {R}^{+} {ω}_{b} {t}_{a} ) ,$$

$${F}_{ab}^{( 3 )} = \frac{1}{2} ( {t}_{b} R ) ( {R}^{+} {ω}_{a} R ) - \frac{1}{2} ( {t}_{a} R ) ( {R}^{+} {ω}_{b} R ) + ( {R}^{+} {ω}_{a} {ω}_{b} R ) ,$$

$${F}_{ab}^{( 4 )} = \frac{1}{4} ( {R}^{+} {ω}_{a} R ) ( {R}^{+} {ω}_{b} R ) .$$

and potentials φ^{{k}}_{ab} satisfy the recurrence relations:

$${φ}_{ab}^{( k + 1 )} ( R ) = \frac{1}{R} \frac{d}{dR} {φ}_{ab}^{( k )} ( R ) , k = 0 , 1 , … \qquad ( B4 )$$

The ES potential U^{ES} between the two clouds is written as:

U_{ab}^{ES}(R_{ab})=332.064·qₐq_{b}φ_{ab}(R_{ab})

The potentials are in kcal/mol, the components of vectors R_{ab} and t are in Å, charges are in a.u. This formula is also valid for core-cloud and core-core interactions by replacing the cloud charges with the core charges, nullifying vectors t and tensor ω for the core, with the corresponding width parameter(s) formally approaching zero.

### Exchange Potential

The EX potential U^{EX} is written similarly to that of ES:

U_{ab}^{EX}=332.064·C_{A}^{EX}C_{B}^{EX}χ_{ab}(R_{ab}) (B6)

where C^{EX}_{A}, C^{EX}_{B} are the force parameters for atom types ‘A’ and ‘B’, and

$${χ}_{ab} ( {R}_{ab} , {t}_{a} , {t}_{b} ) = {\sum }_{k = 0}^{4} {F}_{ab}^{( k )} ( {R}_{ab} , {t}_{a} , {t}_{b} ) {χ}_{ab}^{( k )} ( {R}_{ab} ) \qquad ( B7 )$$

The algebraic factors F were defined in Eq. (B4), potentials χ^{{k}}_{ab}(R_{ab}) satisfy the recurrence relations in Eq. (B5)

$${χ}_{ab}^{( 0 )} ( r ) ≡ {u}_{A} {u}_{B} \frac{{e}^{- r / {u}_{B}} - {e}^{- r / {u}_{A}}}{( {u}_{B} - {u}_{A} ) r} \qquad ( B8 )$$

where u_{A} and u_{B} are the EX width parameters that generally differ from ES parameters w_{A} and w_{B}. Again, χ_{ab}⁽⁰⁾ represents a functional form of the exchange potential.

### Dispersion Potential

U_{ab}^{DS}=−332.064·((C_{A}^{DS6}C_{B}^{DS6}ψ₆(R_{ab})+C_{A}^{DS8}C_{B}^{DS8}ψ_{S}(R_{ab})) (B9)

where C^{DS6}, C_{DS8} are the force constants and ψ's are the Tang-Toennies's functions:

$${ψ}_{n} ( r ) = {r}^{- n} [ 1 - {e}^{- 2 r / ( {v}_{A} + {v}_{B} )} {\sum }_{k = 1}^{n} \frac{1}{k !} {( \frac{2 r}{{v}_{A} + {v}_{B}} )}^{k} ] \qquad ( B10 )$$

v_{A} and v_{B} being the dispersion width parameters that generally differ from those for electrostatics and exchange.

### Induction Potential

The corresponding anharmonic restraint potential for the atom ‘a’ being represented as:

$${U}_{a}^{IN} ( {τ}_{a} ) = 2240.88 \cdot \frac{{( {q}_{a} {t}_{A}^{max} )}^{2}}{{α}_{A} {M}_{a}} \frac{{τ}_{a}^{2} + {\sum }_{b ∈ \{ a \}} {{s}_{AB} ( {τ}_{a} {n}_{ab} )}^{2}}{1 + \sqrt{1 - {( {τ}_{a} )}^{2}}} , {M}_{a} = 1 + \frac{1}{3} {\sum }_{b ∈ \{ a \}} {s}_{AB} \qquad ( B11 )$$

where α_{A} is the QM-parameterized force field parameter characterizing polarizability prescribed to atom-type A while bond-type parameters s_{AB} describe the anisotropy of the restraint potential (note that generally s_{AB}≠s_{BA}), the vector τₐ was introduced in Eq. (A6). Note that the force due to potential in Eq. (B12) approaches infinity as τₐ approaches 1. This peculiarity of the force field model restraint potential avoids the polarization catastrophe and provides the existence of a solution of the problem of optimization of electron density in any physically reasonable external field.

C) Valence Interactions

he Hamiltonian of valence interaction is given in the form:

H=H^{BS}+H^{AB}+H^{StBn}+H^{OOP}+H^{TORS} (C1)

where the terms correspond respectively to bond stretching (BS), angle bending (AB), stretch-bend (StBn), out-of-plane bending (OOP), and torsion interactions (TORS).

The bond stretching component is of the form:

$${H}^{BS} = \frac{1}{2} \sum {k}_{IJ}^{( b )} Δ {r}_{ij}^{2} ( 1 + {c}_{b} Δ {r}_{ij} + \frac{7}{12} {c}_{b}^{2} Δ {r}_{ij}^{2} ) \qquad ( C2 )$$

where

Δrᵢⱼ=|rᵢ−rⱼ|−f_{IJ}⁽⁰⁾ (C3)

In Eq. (C2), the sum is over all the bond pairs, I and J are the indices of the atomic types ascribed respectively to atoms i and j, r_{IJ}⁽⁰⁾ and k_{IJ}^{(b)} are respectively the equilibrium (or reference) bond length and the bond force constant determined by the atomic type pair and the specific bond type index, and c_{b} is a model parameter.

The angle bending term is given by:

$${H}^{AB} = \frac{1}{2} \sum {k}_{IJK}^{( a )} {Δφ}_{ijk}^{2} ( 1 + {c}_{a} {Δφ}_{ijk} ) \qquad ( C4 )$$

where the summation is performed over all bend triplets i-j, j-k, the indices I, J and K corresponding to the atomic types ascribed respectively to atoms i, j and k. Other notations are:

Δφᵢⱼₖ=φᵢⱼₖ−φ_{IJK}⁽⁰⁾ (C5)

where φᵢⱼₖ is the angle between i-j and k-j bonds, φ_{IJK}^{{0}} and k_{IJK}^{{a}} are the equilibrium (or reference) angle and the force constant determined by the atomic type triplet and the specific angle type index, and cₐ is a model parameter.

Stretch-bend interaction is given by:

H^{St-Bn}=ΣΔφᵢⱼₖ(k_{IJK}^{(ba)}Δrᵢⱼ+k_{KJI}^{(ba)}Δrₖⱼ) (C6)

where the summation is similar to Eq. (C4). The bond length and angle deviations, Δrᵢⱼ, Δrₖⱼ and Δφᵢⱼₖ are defined by Eqs. (C3) and (C5), k^{(ba)}_{IJK} and k^{(ba)}_{KJI} are the force constants for the interaction of ij and kj bond-angle, respectively. These constants are determined by the atomic type triplet and the specific stretch-bend type index.

Out-of-plane interaction is given by:

$${H}^{OOP} = \frac{1}{2} \sum {k}_{LIKL}^{( oop )} ( {Δχ}_{ikl}^{2} + {Δχ}_{kil}^{2} + {Δχ}_{lik}^{2} ) \qquad ( C7 )$$

where the sum is over all bond tetrahedrals i-j-k-l with the 3-bond central (or vertex) atom j and the bonds i-j, k-j, l-j, the indices I, J, K and L correspond to the atomic types ascribed respectively to atoms i, j, k, and l. The angle deviation Δχᵢₖₗ is defined as the angle between the vector (rᵢ−rⱼ) and the plane defined by vectors (rₖ−rₗ) and (rₗ−rⱼ); that is

$$sin ( {Δχ}_{ikl} ) = \frac{{n}_{ij} \cdot ( {n}_{kj} \times {n}_{lj} )}{\vert {n}_{kj} \times {n}_{lj} \vert} \qquad ( C8 )$$

where

nₐⱼ=(rₐ−rⱼ)/|rₐ−rⱼ|, a=i,k,l (C9)

The force constant k_{IJKL} is determined by the atomic type quartet IJKL.

The torsion component is of the form

H^{TORS}=1/2Σ[V_{IJKL}⁽¹⁾(1+cos ϕ)+V_{IJKL}⁽²⁾(1−cos 2ϕ)+V_{IJKL}⁽³⁾(1+cos 3ϕ)] (C10)

where the sum is over all bond quartets i-j, j-k, k-l, the indices I, J, K, and L correspond to the atomic types ascribed respectively to atoms i,j, k, and l. ø is a standard torsion angle defined as the angle between the planes i-j-k and j-k-l, given by:

cos ϕ=mᵢⱼ·mₗₖ (C11)

where

$${m}_{ij} = \frac{{n}_{ij} - {n}_{jk} ( {n}_{jk} \cdot {n}_{ij} )}{\sqrt{1 - {( {n}_{ij} \cdot {n}_{jk} )}^{2}}} , {m}_{kl} = \frac{{n}_{kj} - {n}_{jk} ( {n}_{jk} \cdot {n}_{kl} )}{\sqrt{1 - {( {n}_{kl} \cdot {n}_{jk} )}^{2}}} \qquad ( C12 )$$

The force constants V_{IJKL} are determined by the atomic type quartet and two specific indices, the first relating to the type of the central bond j-k, while the second one relates to the hybridization state of atoms j and k.

### Example Parameter Fitting

Monomer, direr, and multimer QM data of high quality is used for fitting the QM-parameterized force field model. From the monomer calculations the ES potential map is used as well as polarizability and its related properties. The stochastic Covariance matrix adaptation evolution strategy (CMA-ES) can be used.

As shown in FIG. 5G, FF:QM differences (or errors or faithfulness) for ethanol-water dimers as a function of closest distance. The FF:QM faithfulness becomes worse in the region of close approach and high electron overlap where the electron densities and corresponding integrals have large values and derivatives. The low-energy dimer (E−−4.5 kcal/mol, ERR=0.9 kcal/mol) is shown in the figure. This configuration can be better described by going to a higher (L=3,4) level of the atomic multipole expansion and/or by including multiple slater exponents, but truncations of L=2 and one exponent for reasons of computational speed and parameter complexity are used.

Multimers of ethanol with 2,3,4 water molecules were extracted from Molecular dynamics simulations with the force field model. Geometries of those multimers were optimized with the mp2/cc-pVTZ method. Nonadditive energies are full nonbonded energy of MP2/CBS (aug-cc-pVTZ->aug-cc-pVQZ extrapolated) minus energies of dimers nonbonded energy.

As expected for ‘formula’-based functional forms, the predictability of the QM-parameterized force field model can saturate at its maximum achievable accuracy. In contrast, machine learning (ML) models can theoretically achieve accuracy, but the training data requirements are linear in log-log (accuracy vs training size) space.

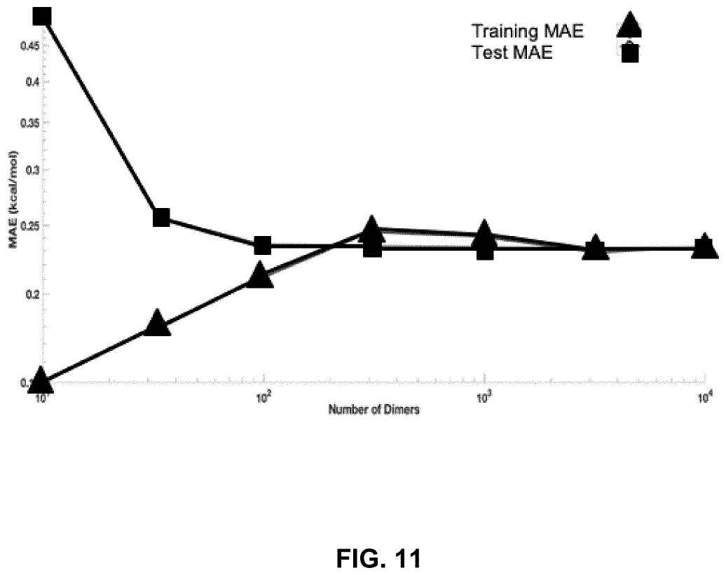

FIG. 11 shows double logarithm learning plot constructed with a subset of the total set of dimers for training data set (triangles as shown in FIG. 11) and testing data set (squares as shown in FIG. 11). This subset includes amides (N-methyl amides, acetamide) and water (9,550 dimers). Randomly selected configurations from this subset are used in increasing numbers to construct the plot. A fixed amount of monomer properties i. e. the electrostatic potential surfaces, polarizability tensor etc. were kept constant. It is clear that the force field optimization and/or improvement converges almost to the point of saturation around 330 dimers for this subset.

Solvation data for various functional groups is given in Table 3. Table 3 shows solvation data with free energies of hydration, solvation and partition coefficients for the various functional groups. Table 4 shows a comparison of QM-parameterized force field model and PIMD calculated free energies with experiment data for neutral amino-acid analogues. Table 5 shows a comparison of hydration free energies as predicted by the QM-parameterized force field model described herein, GAFF, and AMOEBA force fields. Table 6 shows computations of free energies of hydration as predicted by the QM-parameterized force field model described herein, and models performed by academic collaborations.

Comparison with Implicit Solvent Models and Machine Learning Models

Tables 3-6 show comparisons of the free energy of hydration predictions of various functional group molecules based on the QM-parameterized force field model with that of the implicit solvent models namely Solvation Model Dielectric (SMD), Polarizable Continuum Model (PCM), and COSMO/COSMO-RS. Due to unavailability of free energy data for the original COSMO model, the free energies of solvation for the molecule dataset are calculated using MolPro ver. 2012 (gcosmo, epsilon=80.0,df-ks,B3LYP). The MAE for COSMO (computed using MolPro ver. 2012) is 2.5 kcal/mol which is in reasonable agreement with the MAE of ˜2 kcal/mol for COSMO. The comparisons to other implicit solvent models such as SMD, PCM and COSMO-RS are limited to the overlap of the molecules computed using QM-parameterized force field model. The free energies of hydration as predicted by these implicit solvent models (SMD, PCM) were received. For these subset of compounds, as shown in Table 3, the MAE's for the SMD, PCM are 0.61 kcal/mol and 1.49 kcal/mol, respectively. The free energies of hydration as determined by COSMO-RS are calculated and for the dataset the MAE to be 0.42 kcal/mol if calculated.

Machine learning models can be used for free energy determination. Based on an overlapping dataset of free energy computations of neutral organic molecules, the MAE for Free Energy Machine Learning Model (FML) was 0.50 kcal/mol. The MAE's for the free energies of hydration on the subset of neutral organic molecules dataset based on these implicit solvent models and the machine learning model-FML are consistent with the MAE's calculated and received using other models.

TABLE 3
Solvation_data: The data for free energies of hydration, solvation and partition coefficients for the various functional groups.

| | water | | | | | CHEX | | | | | | | |
|---|---|---|---|---|---|---|---|---|---|---|---|---|---|
| | Exp | | | | | Exp | | | | | | | |
| | (hy- | | | | | (sol- | | | | | | | |
| | dration) | | | Arrow | Arrow | vation) | | | Exp | | | Arrow | Arrow |
| | (Kcal/ | Arrow | Arrow | MD | PIMD8 | (Kcal/ | Arrow | ARROW | (log | Arrow | Arrow | MD | PIMD |
| Compounds | mol) | (MD) | (PIMD8) | Error | Error | mol) | (MD) | (PIMD4) | D) | (MD) | (PIMD8) | Error | Error |
| Alkanes | | | | | | | | | | | | | |
| Cyclopropane | 0.75 | 0.45 | 1.04 | 0.30 | 0.29 | | | | | | | | |
| Cyclopentane | 1.20 | 0.49 | 1.32 | 0.71 | 0.12 | | | | | | | | |
| Cyclohexane | 1.23 | 0.20 | 1.13 | 1.03 | 0.10 | −4.42 | −4.23 | −4.02 | 4.14 | 3.25 | 3.78 | 0.89 | 0.36 |
| Cycloheptane | 0.79 | 0.12 | 0.91 | 0.67 | 0.12 | | | | | | | | |
| Isobutane | 2.28 | 1.35 | 2.26 | 0.93 | 0.02 | −2.61 | −2.94 | −2.71 | 3.59 | 3.15 | 3.65 | 0.44 | 0.06 |
| NeoPentane | 2.50 | 1.45 | 2.38 | 1.05 | 0.12 | | | | | | | | |
| Methane | 1.96 | 1.64 | 1.87 | 0.32 | 0.09 | 0.14 | 0.15 | 0.18 | 1.33 | 1.09 | 1.24 | 0.25 | 0.09 |
| Ethane | 1.79 | 1.37 | 1.91 | 0.42 | 0.12 | | | | | | | | |
| Propane | 1.96 | 1.30 | 1.96 | 0.66 | 0.00 | −2.03 | −2.26 | −2.08 | 2.93 | 2.61 | 2.97 | 0.31 | 0.04 |
| Butane | 2.10 | 1.40 | 2.14 | 0.70 | 0.04 | −2.72 | −3.18 | −2.92 | 3.54 | 3.36 | 3.71 | 0.18 | 0.17 |
| Pentane | 2.34 | 1.39 | 2.28 | 0.95 | 0.06 | −3.50 | −3.92 | −3.78 | 4.28 | 3.89 | 4.45 | 0.39 | 0.17 |
| Hexane | 2.51 | 1.57 | 2.45 | 0.94 | 0.06 | | | | | | | | |
| Octane | 2.89 | 1.63 | 2.84 | 1.27 | 0.06 | −5.63 | −6.22 | −5.96 | 6.25 | 5.75 | 6.45 | 0.50 | 0.20 |
| Alkenes | | | | | | | | | | | | | |
| Ethene | 1.28 | 0.99 | 1.47 | 0.29 | 0.19 | | | | | | | | |
| Propene | 1.32 | 0.84 | 1.37 | 0.48 | 0.05 | | | | | | | | |
| Buta-13-diene | 0.61 | 0.30 | 0.96 | 0.31 | 0.35 | | | | | | | | |
| But-1-ene | 1.38 | 0.84 | 1.63 | 0.54 | 0.25 | | | | | | | | |
| pent-1-ene | 1.68 | 1.09 | 1.93 | 0.59 | 0.25 | | | | | | | | |
| Amines | | | | | | | | | | | | | |
| methylamine | −4.55 | −5.04 | −4.39 | 0.49 | 0.16 | | | | | | | | |
| dimethylamine | −4.30 | −5.68 | −4.53 | 1.38 | 0.23 | | | | | | | | |
| trimethylamine | −3.20 | −4.24 | −3.18 | 1.04 | 0.02 | −2.63 | −3.54 | −3.19 | −0.42 | −0.51 | 0.01 | 0.10 | 0.43 |
| butanamine | −4.24 | −5.41 | −3.96 | 1.17 | 0.28 | −3.92 | −3.98 | −3.86 | −0.23 | −1.05 | −0.07 | 0.81 | 0.16 |
| ethylamine | −4.50 | −5.53 | −4.38 | 1.03 | 0.12 | −2.13 | −2.23 | −2.12 | −1.74 | −2.42 | −1.65 | 0.69 | 0.08 |
| Ethers | | | | | | | | | | | | | |
| DiMethylEther | −1.91 | −2.78 | −2.28 | 0.87 | 0.37 | | | | | | | | |
| DiethylEther | −1.75 | −2.70 | −1.85 | 0.95 | 0.10 | | | | | | | | |
| DiMethoxyEthane | −4.84 | −5.21 | −4.64 | 0.37 | 0.20 | | | | | | | | |
| MethylPropylEther | −1.66 | −2.32 | −1.66 | 0.66 | 0.00 | | | | | | | | |
| Aldehydes | | | | | | | | | | | | | |
| acetaldehyde | −3.50 | −3.68 | −3.23 | 0.18 | 0.27 | | | | | | | | |
| propionaldehyde | −3.43 | −3.73 | −3.18 | 0.30 | 0.25 | | | | | | | | |
| buytyraldehyde | −3.18 | −3.79 | −3.25 | 0.61 | 0.07 | | | | | | | | |
| Ketones | | | | | | | | | | | | | |
| Acetone | −3.80 | −4.13 | −3.49 | 0.33 | 0.31 | −2.67 | −2.99 | −2.78 | −0.83 | −0.84 | −0.52 | 0.01 | 0.31 |
| 2-Butanone | −3.71 | −4.14 | −3.55 | 0.43 | 0.16 | −3.47 | −3.75 | −3.60 | −0.18 | −0.29 | 0.04 | 0.11 | 0.21 |
| 2-Pentanone | −3.52 | −4.19 | −3.51 | 0.67 | 0.01 | −4.19 | −4.57 | −4.45 | 0.49 | 0.27 | 0.69 | 0.22 | 0.20 |
| 3-Pentanone | −3.41 | −4.29 | −3.64 | 0.88 | 0.23 | −4.30 | −4.75 | −4.56 | 0.65 | 0.34 | 0.68 | 0.32 | 0.02 |
| hexa2none | −3.28 | −3.97 | −3.48 | 0.69 | 0.20 | −4.77 | −5.56 | −5.13 | 1.09 | 1.16 | 1.21 | 0.07 | 0.11 |
| Acids | | | | | | | | | | | | | |
| AceticAcid | −6.69 | −6.88 | −6.35 | 0.19 | 0.34 | −2.20 | −2.29 | −2.19 | −3.29 | −3.36 | −3.05 | 0.07 | 0.25 |
| propionicacid | −6.46 | −6.70 | −6.21 | 0.24 | 0.25 | −3.32 | −3.26 | −3.14 | −2.30 | −2.52 | −2.25 | 0.22 | 0.05 |
| butyricacid | −6.35 | −6.60 | −5.95 | 0.25 | 0.40 | | | | | | | | |
| Aromatics | | | | | | | | | | | | | |
| Benzene | −0.88 | −1.46 | −1.05 | 0.58 | 0.17 | −4.19 | −4.87 | −4.52 | 2.43 | 2.50 | 2.55 | 0.08 | 0.12 |
| Napthalene | −2.40 | −3.14 | −2.64 | 0.74 | 0.24 | −7.17 | −7.64 | −7.43 | 3.50 | 3.30 | 3.51 | 0.19 | 0.01 |
| Anthracene | −3.95 | −4.27 | −3.88 | 0.32 | 0.07 | | | | | | | | |
| tetracene | −5.5 | −5.89 | −5.06 | 0.39 | 0.44 | | | | | | | | |
| pyrene | −4.52 | −4.97 | −4.51 | 0.45 | 0.01 | | | | | | | | |
| phenanthrene | −3.88 | −4.23 | −3.97 | 0.35 | 0.09 | | | | | | | | |
| Biphenyl | −2.66 | −3.83 | −2.79 | 1.17 | 0.13 | | | | | | | | |
| Toluene | −0.89 | −1.72 | −0.99 | 0.83 | 0.10 | −4.90 | −5.84 | −5.52 | 2.94 | 3.02 | 3.32 | 0.08 | 0.38 |
| Phenol | −6.62 | −7.64 | −6.80 | 1.02 | 0.18 | −5.56 | −5.69 | −5.46 | −0.78 | −1.43 | −0.98 | 0.65 | 0.21 |
| p-cresol | −6.14 | −7.62 | −6.71 | 1.48 | 0.57 | −5.89 | −6.37 | −6.31 | −0.18 | −0.92 | −0.29 | 0.73 | 0.11 |
| m-cresol | −5.49 | −7.61 | −6.52 | 2.12 | 1.03 | −5.20 | −6.43 | −6.10 | −0.21 | −0.86 | −0.31 | 0.65 | 0.09 |
| o-cresol | −5.87 | −7.14 | −6.15 | 1.27 | 0.28 | −6.02 | −6.38 | −6.11 | 0.11 | −0.56 | −0.03 | 0.67 | 0.14 |
| Water | −6.30 | −6.81 | −6.13 | 0.51 | 0.17 | −0.39 | −0.47 | −0.45 | −4.33 | −4.65 | −4.17 | 0.31 | 0.17 |
| Alcohols | | | | | | | | | | | | | |
| Methanol | −5.11 | −5.39 | −4.76 | 0.28 | 0.35 | −1.29 | −1.71 | −1.53 | −2.80 | −2.70 | −2.37 | 0.10 | 0.43 |
| Ethanol | −5.01 | −5.75 | −4.94 | 0.74 | 0.07 | −2.59 | −2.64 | −2.47 | −1.77 | −2.28 | −1.81 | 0.50 | 0.04 |
| Propanol | −4.92 | −5.50 | −4.74 | 0.58 | 0.18 | −2.73 | −3.45 | −3.13 | −1.61 | −1.50 | −1.18 | 0.10 | 0.43 |
| 2-Propanol | −4.62 | −5.88 | −4.62 | 1.26 | 0.00 | −2.37 | −3.37 | −3.04 | −1.65 | −1.84 | −1.16 | 0.19 | 0.49 |
| 1-Butanol | −4.72 | −5.57 | −4.66 | 0.85 | 0.06 | −3.52 | −4.42 | −4.04 | −0.88 | −0.85 | −0.46 | 0.03 | 0.42 |
| 2-Butanol | −4.58 | −5.99 | −4.66 | 1.41 | 0.08 | | | | | | | | |
| 2-Methyl-2- | −4.51 | −5.36 | −4.16 | 0.85 | 0.35 | | | | | | | | |
| Propanol | | | | | | | | | | | | | |
| 1-Pentanol | −4.47 | −5.65 | −4.52 | 1.18 | 0.05 | | | | | | | | |
| 2-Methyl-2- | −4.43 | −5.36 | −4.60 | 0.93 | 0.17 | | | | | | | | |
| Butanol | | | | | | | | | | | | | |
| 2-Pentanol | −4.39 | −6.07 | −4.47 | 1.68 | 0.08 | | | | | | | | |
| 3-Pentanol | −4.35 | −5.71 | −4.20 | 1.36 | 0.15 | | | | | | | | |
| Cyclohexanol | −5.47 | −6.56 | −5.13 | 1.09 | 0.34 | | | | | | | | |
| Octanol | −4.09 | −5.49 | −3.87 | 1.40 | 0.22 | | | | | | | | |
| Esters | | | | | | | | | | | | | |
| EthylAcetate | −3.12 | −3.40 | −2.80 | 0.28 | 0.32 | −3.55 | −3.20 | −3.05 | 0.32 | −0.14 | 0.19 | 0.46 | 0.13 |
| MethylPropionate | −2.93 | −3.55 | −3.03 | 0.62 | 0.10 | −3.71 | −3.19 | −3.19 | 0.57 | −0.27 | 0.12 | 0.84 | 0.45 |
| PropylAcetate | −2.86 | −3.50 | −2.79 | 0.64 | 0.07 | −4.36 | −4.12 | −3.79 | 1.10 | 0.45 | 0.73 | 0.65 | 0.37 |
| ButylAcetate | −2.55 | −3.40 | −2.48 | 0.85 | 0.07 | −4.94 | 4.98 | −4.79 | 1.75 | 1.16 | 1.70 | 0.60 | 0.06 |
| Amides | | | | | | | | | | | | | |
| Acetamide | −9.71 | −10.58 | −9.65 | 0.87 | 0.06 | −3.01 | −3.64 | −3.59 | −4.91 | −5.09 | −4.44 | 0.17 | 0.47 |
| N- | −10 | −11.52 | −10.49 | 1.52 | 0.49 | | | | | | | | |
| Methylacetamide | | | | | | | | | | | | | |
| DiMethylAcetamide | −8.5 | −9.14 | −8.51 | 0.64 | 0.01 | | | | | | | | |
| propionamide | −9.4 | −10.61 | −9.57 | 1.21 | 0.17 | | | | | | | | |
| Thiols | | | | | | | | | | | | | |
| MethaneThiol | −1.219 | −2.01 | −1.65 | 0.79 | 0.43 | | | | | | | | |
| EthaneThiol | −1.24 | −2.18 | −1.67 | 0.94 | 0.43 | | | | | | | | |
| Propanethiol | −1.05 | −1.86 | −1.45 | 0.81 | 0.40 | −3.12 | −4.44 | −4.01 | 1.52 | 1.89 | 1.87 | 0.38 | 0.36 |
| 1-Butanethiol | −0.99 | −2.14 | −1.49 | 1.15 | 0.50 | | | | | | | | |
| Sulfides | | | | | | | | | | | | | |
| DiMethylSulfide | −1.54 | −2.13 | −1.70 | 0.59 | 0.16 | | | | | | | | |
| DiEthylSulfide | −1.43 | −2.32 | −1.55 | 0.89 | 0.12 | | | | | | | | |
| EthylMethylSulfide | −1.5 | −1.97 | −1.61 | 0.47 | 0.11 | −3.78 | −4.61 | −4.29 | 1.67 | 1.94 | 1.96 | 0.27 | 0.29 |
| DimethylDisulfide | −1.83 | −2.64 | −1.85 | 0.81 | 0.02 | | | | | | | | |
| Heterorings | | | | | | | | | | | | | |
| 3-methyl-indole | −5.88 | −7.88 | −6.90 | 2.00 | 1.02 | −8.08 | −8.32 | −8.07 | 1.61 | 0.32 | 0.86 | 1.29 | 0.76 |
| Aniline | −5.54 | −5.71 | −5.48 | 0.17 | 0.06 | −5.52 | −5.94 | −5.64 | −0.01 | 0.17 | 0.12 | 0.18 | 0.13 |
| Imidazole | −9.6 | −10.41 | −9.89 | 0.81 | 0.29 | | | | | | | | |
| Pyrrole | −4.8 | −4.72 | −4.17 | 0.08 | 0.63 | | | | | | | | |
| pyridine | −4.69 | −5.53 | −4.77 | 0.84 | 0.08 | −4.30 | −4.67 | −4.41 | −0.29 | −0.63 | −0.26 | 0.34 | 0.03 |

TABLE 4
Comparison of QM-parameterized force field model and PIMD free
energies to experiment for the neutral amino-acid analogues.

| Neutral | water | | Exp | Arrow | Arrow | Neutral | CHEX | | Exp |
|---|---|---|---|---|---|---|---|---|---|
| amino-acid | Arrow | Arrow | (hy- | MD | PIMD8 | amino-acid | Arrow | Arrow | (sol- |
| analogues | (MD) | (PIMD8) | dration) | Error | Error | analogues | (MD) | (PIMD4) | vation) |
| Isobutane | 1.35 | 2.26 | 2.28 | 0.93 | 0.02 | Isobutane | −2.94 | −2.71 | −2.61 |
| (Leu) | | | | | | (Leu) | | | |
| Methane | 1.64 | 1.87 | 1.96 | 0.32 | 0.09 | Methane | 0.15 | 0.18 | 0.14 |
| (Ala) | | | | | | (Ala) | | | |
| Propane | 1.30 | 1.96 | 1.96 | 0.66 | 0.00 | Propane | −2.26 | −2.08 | −2.03 |
| (Val) | | | | | | (Val) | | | |
| Butane (Ile) | 1.40 | 2.14 | 2.10 | 0.70 | 0.04 | Butane (Ile) | −3.18 | −2.92 | −2.72 |
| n- | −5.41 | −3.96 | −4.24 | 1.17 | 0.28 | n- | −3.98 | −3.86 | −3.92 |
| butanamine | | | | | | butanamine | | | |
| (Lys) | | | | | | (Lys) | | | |
| Acetic acid | −6.88 | −6.35 | −6.69 | 0.19 | 0.34 | Acetic acid | −2.29 | −2.19 | −2.20 |
| (Asp) | | | | | | (Asp) | | | |
| Propionic | −6.70 | −6.21 | −6.46 | 0.24 | 0.25 | Propionic | −3.26 | −3.14 | −3.32 |
| acid (Glu) | | | | | | acid (Glu) | | | |
| Toluene | −1.72 | −0.99 | −0.89 | 0.83 | 0.10 | Toluene | −5.84 | −5.52 | −4.90 |
| (Phe) | | | | | | (Phe) | | | |
| p-Cresol | −7.62 | −6.71 | −6.14 | 1.48 | 0.57 | p-Cresol | −6.37 | −6.31 | −5.89 |
| (Tyr) | | | | | | (Tyr) | | | |
| Methanol | −5.39 | −4.76 | −5.11 | 0.28 | 0.35 | Methanol | −1.71 | −1.53 | −1.29 |
| (Ser) | | | | | | (Ser) | | | |
| Ethanol | −5.75 | −4.94 | −5.01 | 0.74 | 0.07 | Ethanol | −2.64 | −2.47 | −2.59 |
| (Thr) | | | | | | (Thr) | | | |
| Acetamide | −10.58 | −9.65 | −9.71 | 0.87 | 0.06 | Acetamide | −3.64 | −3.59 | −3.01 |
| (Asn) | | | | | | (Asn) | | | |
| Propionamide | −10.61 | −9.57 | −9.4 | 1.21 | 0.17 | Propionamide | −4.25 | −4.11 | −3.77 |
| (Gln) | | | | | | (Gln) | | | |
| Methanethiol | −2.01 | −1.65 | −1.219 | 0.79 | \ | Methanethiol | −2.85 | −2.76 | −2.46 |
| (Cys) | | | | | | (Cys) | | | |
| Methyl- | −1.97 | −1.61 | −1.5 | 0.47 | 0.11 | Methyl- | −4.61 | −4.29 | −3.78 |
| ethylsulfide | | | | | | ethylsulfide | | | |
| (Met) | | | | | | (Met) | | | |
| Methylindole | −7.88 | −6.90 | −5.88 | 2.00 | 1.02 | Methylindole | −8.32 | −8.07 | −8.08 |
| (Trp) | | | | | | (Trp) | | | |
| Methylimidazole | −10.46 | −10.09 | −10.06 | 0.40 | 0.03 | Methylimidazole | −5.92 | −5.68 | −5.59 |
| (His) | | | | | | (His) | | | |

| | | | Neutral | | | | | |
|---|---|---|---|---|---|---|---|---|
| Neutral | Arrow | Arrow | amino- | | | logD | Arrow | Arrow |
| amino-acid | MD | PIMD4 | acid | Arrow | Arrow | Exp | MD | PIMD |
| analogues | Error | Error | analogues | (MD) | (PIMD) | (logD) | Error | Error |
| Isobutane | 0.33 | 0.10 | Isobutane | 3.15 | 3.65 | 3.59 | 0.44 | 0.06 |
| (Leu) | | | (Leu) | | | | | |
| Methane | 0.01 | 0.04 | Methane | 1.09 | 1.24 | 1.33 | 0.25 | 0.09 |
| (Ala) | | | (Ala) | | | | | |
| Propane | 0.23 | 0.05 | Propane | 2.61 | 2.97 | 2.93 | 0.31 | 0.04 |
| (Val) | | | (Val) | | | | | |
| Butane (Ile) | 0.46 | 0.20 | Butane (Ile) | 3.36 | 3.71 | 3.54 | 0.18 | 0.17 |
| n- | 0.06 | 0.06 | n- | −1.05 | −0.07 | −0.23 | 0.81 | 0.16 |
| butanamine | | | butanamine | | | | | |
| (Lys) | | | (Lys) | | | | | |
| Acetic acid | 0.09 | 0.01 | Acetic acid | −3.36 | −3.05 | −3.29 | 0.07 | 0.25 |
| (Asp) | | | (Asp) | | | | | |
| Propionic | 0.06 | 0.18 | Propionic | −2.52 | −2.25 | −2.30 | 0.22 | 0.05 |
| acid (Glu) | | | acid (Glu) | | | | | |
| Toluene | 0.94 | 0.62 | Toluene | 3.02 | 3.32 | 2.94 | 0.08 | 0.38 |
| (Phe) | | | (Phe) | | | | | |
| p-Cresol | 0.48 | 0.42 | p-Cresol | −0.92 | −0.29 | −0.18 | 0.73 | 0.11 |
| (Tyr) | | | (Tyr) | | | | | |
| Methanol | 0.42 | 0.24 | Methanol | −2.70 | −2.37 | −2.80 | 0.10 | 0.43 |
| (Ser) | | | (Ser) | | | | | |
| Ethanol | 0.05 | 0.12 | Ethanol | −2.28 | −1.81 | −1.77 | 0.50 | 0.04 |
| (Thr) | | | (Thr) | | | | | |
| Acetamide | 0.63 | 0.58 | Acetamide | −5.09 | −4.44 | 4.91 | 0.17 | 0.47 |
| (Asn) | | | (Asn) | | | | | |
| Propionamide | 0.48 | 0.34 | Propionamide | −4.67 | −4.01 | −4.13 | 0.54 | 0.12 |
| (Gln) | | | (Gln) | | | | | |
| Methanethiol | 0.39 | 0.30 | Methanethiol | 0.62 | 0.82 | 0.91 | 0.29 | 0.09 |
| (Cys) | | | (Cys) | | | | | |
| Methyl- | 0.83 | 0.51 | Methyl- | 1.94 | 1.96 | 1.67 | 0.27 | 0.29 |
| ethylsulfide | | | ethylsulfide | | | | | |
| (Met) | | | (Met) | | | | | |
| Methylindole | 0.24 | 0.01 | Methylindole | 0.32 | 0.86 | 1.61 | 1.29 | 0.76 |
| (Trp) | | | (Trp) | | | | | |
| Methylimidazole | 0.33 | 0.09 | Methylimidazole | −3.33 | −3.23 | −3.28 | 0.05 | 0.04 |
| (His) | | | (His) | | | | | |

TABLE 5
Comparison of free energies of hydration between QM-parameterized
force field model, GAFF, and AMOEBA for select compounds. The
GAFF and AMOEBA predictions are taken from literature.

| | Exp | Arrow | | | Arrow | GAFF | AMOEBA |
|---|---|---|---|---|---|---|---|
| Compounds | (hydration) | (PIMD8) | GAFF | AMOEBA | error | error | error |
| Alkanes | | | | | | | |
| Methane | 1.96 | 1.87 | 2.20 | 1.73 | 0.09 | 0.24 | 0.23 |
| Ethane | 1.79 | 1.91 | 2.58 | 1.73 | 0.12 | 0.79 | 0.06 |
| Propane | 1.96 | 1.96 | 2.56 | 1.69 | 0.00 | 0.60 | 0.27 |
| Butane | 2.10 | 2.14 | 2.54 | 1.11 | 0.04 | 0.44 | 0.99 |
| Octane | 2.88 | 2.84 | 3.13 | 1.74 | 0.04 | 0.25 | 1.14 |
| Alkenes | | | | | | | |
| But-1-ene | 1.38 | 1.63 | 2.48 | 0.83 | 0.25 | 1.10 | 0.55 |
| Amines | | | | | | | |
| dimethylamine | −4.30 | −4.53 | −3.11 | −3.04 | 0.23 | 1.19 | 1.26 |
| trimethylamine | −3.20 | −3.18 | −2.32 | −2.09 | 0.02 | 0.88 | 1.11 |
| ethylamine | −4.50 | −4.38 | −3.14 | −4.33 | 0.12 | 1.36 | 0.17 |
| Ethers | | | | | | | |
| DiMethylEther | −1.91 | −2.28 | −0.85 | −2.22 | 0.37 | 1.06 | 0.31 |
| Aldehydes | | | | | | | |
| propionaldehyde | −3.43 | −3.88 | −3.41 | −3.08 | 0.45 | 0.02 | 0.35 |
| Acids | | | | | | | |
| AceticAcid | −6.69 | −6.35 | −5.95 | −5.63 | 0.34 | 0.74 | 1.06 |
| Aromatics | | | | | | | |
| Benzene | −0.88 | −1.05 | −0.70 | −0.86 | 0.17 | 0.18 | 0.02 |
| Toluene | −0.89 | −0.99 | −0.71 | −1.53 | 0.10 | 0.18 | 0.64 |
| Phenol | −6.62 | −6.80 | −5.67 | −5.05 | 0.18 | 0.95 | 1.57 |
| p-cresol | −6.14 | −6.71 | −5.36 | −5.60 | 0.57 | 0.78 | 0.54 |
| Water | −6.30 | −6.13 | −6.04 | −5.86 | 0.17 | 0.26 | 0.44 |
| Alcohols | | | | | | | |
| Methanol | −5.11 | −4.78 | −4.37 | −4.79 | 0.33 | 0.74 | 0.32 |
| Ethanol | −5.01 | −4.94 | −3.45 | −4.69 | 0.07 | 1.56 | 0.32 |
| Propanol | −4.92 | −4.74 | −3.12 | −4.85 | 0.18 | 1.80 | 0.07 |
| 2-Propanol | −4.62 | −4.62 | −3.28 | −4.21 | 0.00 | 1.34 | 0.41 |
| Octanol | −4.09 | −3.87 | −5.20 | −2.65 | 0.22 | 1.11 | 1.44 |
| Esters | | | | | | | |
| Methylester | −3.13 | −3.06 | −4.19 | −3.73 | 0.07 | 1.06 | 0.60 |
| Amides | | | | | | | |
| Acetamide | −9.71 | −9.65 | −7.80 | −9.30 | 0.06 | 1.91 | 0.41 |
| N-Methylacetamide | −10.00 | −10.49 | −8.39 | −8.66 | 0.49 | 1.61 | 1.34 |
| Thiols | | | | | | | |
| MethaneThiol | −1.24 | −1.67 | −0.83 | −0.26 | 0.43 | 0.41 | 0.98 |
| 1-Butanethiol | −0.99 | −1.49 | −1.68 | −0.12 | 0.50 | 0.69 | 0.87 |
| Sulfides | | | | | | | |
| DiMethylSulfide | −1.54 | −1.70 | −1.85 | −1.85 | 0.16 | 0.31 | 0.31 |
| EthylMethylSulfide | −1.50 | −1.61 | 0.34 | −1.98 | 0.11 | 1.84 | 0.48 |
| DimethylDisulfide | −1.83 | −1.85 | −1.24 | 1.48 | 0.02 | 0.59 | 3.31 |
| Heterorings | | | | | | | |
| Aniline | −5.54 | −5.48 | −5.92 | −4.78 | 0.06 | 0.38 | 0.76 |
| Imidazole | −9.60 | −9.89 | −7.85 | −10.25 | 0.29 | 1.75 | 0.65 |
| Pyrrole | −4.80 | −4.17 | −3.87 | −3.80 | 0.63 | 0.93 | 1.00 |
| pyridine | −4.69 | −4.77 | −3.45 | −5.59 | 0.08 | 1.24 | 0.90 |

TABLE 6
Free energy comparisons between QM-parameterized force field model, COSMO, SMD, PCM, and FML.

| | Exp | | | | | | | | | | | | |
|---|---|---|---|---|---|---|---|---|---|---|---|---|---|
| | (hy- | | Arrow | | COSMO/ | | | | | | | | |
| | dration) | Arrow | PIMD8 | COSMO/ | Molpro | | SMD | | PCM | | FML | COSMO- | |
| Compounds | (Kcal/mol) | (PIMD8) | Error | Molpro | Error | SMD | Error | PCM | Error | FML | error | RS | error |
| Alkanes | | | | | | | | | | | | | |
| Cyclopropane | 0.75 | 1.04 | 0.29 | −1.30 | 2.05 | 0.10 | 0.65 | −0.60 | 1.35 | −0.01 | 0.76 | 0.64 | 0.40 |
| Cyctopentane | 1.20 | 1.32 | 0.12 | −0.63 | 1.83 | 1.30 | 0.10 | −0.20 | 1.40 | 0.88 | 0.32 | 1.15 | 0.17 |
| Cyclohexane | 1.23 | 1.13 | 0.10 | −0.48 | 1.71 | 1.40 | 0.17 | −0.10 | 1.33 | | | 1.23 | 0.10 |
| Cycloheptane | 0.79 | 0.91 | 0.12 | −0.53 | 1.32 | | | | | | | | |
| Isobutane | 2.28 | 2.26 | 0.02 | | 2.28 | 2.00 | 0.28 | −0.20 | 2.48 | 2.16 | 0.12 | 2.12 | 0.14 |
| NeoPentane | 2.50 | 2.38 | 0.12 | −0.75 | 3.25 | 2.20 | 0.30 | −0.30 | 2.80 | | | | |
| Methane | 1.96 | 1.87 | 0.09 | −0.39 | 2.35 | 2.20 | 0.24 | −0.10 | 2.06 | 0.37 | 1.59 | 1.67 | 0.20 |
| Ethane | 1.79 | 1.91 | 0.12 | −0.40 | 2.19 | 1.80 | 0.01 | −0.10 | 1.89 | 0.94 | 0.85 | 1.89 | 0.02 |
| Propane | 1.96 | 1.96 | 0.00 | −0.48 | 2.43 | 1.80 | 0.16 | −0.10 | 2.06 | 1.46 | 0.50 | 2.01 | 0.05 |
| Butane | 2.10 | 2.14 | 0.04 | −0.55 | 2.65 | 2.00 | 0.10 | −0.20 | 2.30 | | | 2.17 | 0.03 |
| Pentane | 2.34 | 2.28 | 0.06 | −0.63 | 2.97 | 2.20 | 0.14 | −0.20 | 2.54 | | | 2.28 | 0.00 |
| Hexane | 2.51 | 2.45 | 0.06 | −0.86 | 3.37 | 2.40 | 0.11 | −0.20 | 2.71 | | | 2.39 | 0.06 |
| Octane | 2.89 | 2.84 | 0.06 | −0.84 | 3.74 | 2.70 | 0.19 | −0.30 | 3.19 | | | 2.74 | 0.10 |
| Alkenes | | | | | | | | | | | | | |
| Ethene | 1.28 | 1.47 | 0.19 | −1.67 | 2.95 | 1.40 | 0.12 | −0.80 | 2.08 | | | 0.92 | 0.55 |
| Propene | 1.32 | 1.37 | 0.05 | −1.68 | 3.00 | 1.20 | 0.12 | −0.80 | 2.12 | | | 1.05 | 0.32 |
| Buta-13-diene | 0.61 | 0.96 | 0.35 | −2.67 | 3.28 | 0.90 | 0.29 | −1.30 | 1.91 | | | 0.49 | 0.47 |
| But-1-ene | 1.38 | 1.63 | 0.25 | −1.76 | 3.14 | 1.20 | 0.18 | −0.80 | 2.18 | | | 1.27 | 0.36 |
| pent-1-ene | 1.68 | 1.93 | 0.25 | −1.89 | 3.57 | 1.40 | 0.28 | −0.80 | 2.48 | | | 1.37 | 0.56 |
| Amines | | | | | | | | | | | | | |
| methylamine | −4.55 | −4.39 | 0.16 | −5.79 | 1.24 | | | | | | | −4.13 | 0.26 |
| dimethylamine | −4.30 | −4.53 | 0.23 | −4.87 | 0.57 | −3.20 | 1.10 | −2.00 | 2.30 | | | −2.41 | 2.12 |
| trimethylamine | −3.20 | −3.18 | 0.02 | −3.50 | 0.30 | −2.50 | 0.70 | −1.30 | 1.90 | | | −0.63 | 2.55 |
| butanamine | −4.24 | −3.96 | 0.28 | −5.79 | 1.55 | −3.50 | 0.74 | −2.80 | 1.44 | | | −3.85 | 0.11 |
| ethylamine | −4.50 | −4.38 | 0.12 | −5.80 | 1.30 | −3.80 | 0.70 | −2.80 | 1.70 | | | −4.08 | 0.30 |
| Ethers | | | | | | | | | | | | | |
| DiMethylEther | −1.91 | −2.28 | 0.37 | −3.90 | 1.99 | −0.60 | 1.31 | −1.80 | 0.11 | | | | |
| DiethylEther | −1.75 | −1.85 | 0.10 | −3.75 | 2.00 | −1.20 | 0.55 | −1.90 | 0.15 | | | | |
| DiMethoxyEthane | −4.84 | −4.64 | 0.20 | −5.73 | 0.89 | −2.00 | 2.84 | −2.70 | 2.14 | | | | |
| MethylPropylEther | −1.66 | −1.66 | 0.00 | −4.85 | 3.19 | −0.50 | 1.16 | −1.80 | 0.14 | | | | |
| Aldehydes | | | | | | | | | | | | | |
| acetaldehyde | −3.50 | −3.23 | 0.27 | −7.47 | 3.97 | −3.20 | 0.30 | −3.70 | 0.20 | −3.47 | 0.03 | −3.41 | 0.18 |
| propionaldehyde | −3.43 | −3.18 | 0.25 | −8.19 | 4.76 | −3.10 | 0.33 | −3.60 | 0.17 | −3.48 | 0.05 | −3.44 | 0.26 |
| buytyraldehyde | −3.18 | −3.25 | 0.07 | −7.65 | 4.47 | −3.00 | 0.18 | −3.70 | 0.52 | | | −3.18 | 0.07 |
| Ketones | | | | | | | | | | | | | 0.00 |
| Acetone | −3.80 | −3.49 | 0.31 | −8.35 | 4.55 | −4.10 | 0.30 | −4.20 | 0.40 | −3.46 | 0.34 | −4.39 | 0.90 |
| 2-Butanone | −3.71 | −3.55 | 0.16 | −7.86 | 4.15 | −3.80 | 0.09 | −3.90 | 0.19 | | | −3.72 | 0.17 |
| 2-Pentanone | −3.52 | −3.51 | 0.01 | −7.74 | 4.22 | −3.70 | 0.18 | −4.00 | 0.48 | −3.23 | 0.29 | −3.58 | 0.07 |
| 3-Pentanone | −3.41 | −3.64 | 0.23 | −6.74 | 3.33 | −3.50 | 0.09 | −3.70 | 0.29 | −3.25 | 0.16 | −3.58 | 0.06 |
| hexa2none | −3.28 | −3.48 | 0.20 | −7.71 | 4.43 | −3.50 | 0.22 | −4.00 | 0.72 | −3.21 | 0.07 | −3.31 | 0.17 |
| Acids | | | | | | | | | | | | | |
| AceticAcid | −6.69 | −6.35 | 0.34 | −9.16 | 2.47 | −5.70 | 0.99 | −4.50 | 2.19 | | | −6.57 | 0.22 |
| propionicacid | −6.46 | −6.21 | 0.25 | −8.49 | 2.03 | −5.50 | 0.96 | −4.30 | 2.16 | | | −6.06 | 0.15 |
| butyricacid | −6.35 | −5.95 | 0.40 | −8.54 | 2.19 | −5.40 | 0.95 | −4.30 | 2.05 | | | −5.84 | 0.11 |
| Aromatics | | | | | | | | | | | | | |
| Benzene | −0.88 | −1.05 | 0.17 | −3.20 | 2.32 | −0.70 | 0.18 | −1.60 | 0.72 | | | −1.04 | 0.01 |
| Napthalene | −2.40 | −2.64 | 0.24 | −4.51 | 2.11 | −1.50 | 0.90 | −2.30 | 0.10 | | | −2.41 | 0.23 |
| Anthracene | −3.95 | −3.88 | 0.07 | −5.72 | 1.77 | −2.20 | 1.75 | −2.90 | 1.05 | | | −3.71 | 0.17 |
| tetracene | −5.50 | −5.06 | 0.44 | −6.93 | 1.43 | | | | | | | | |
| pyrene | −4.52 | −4.51 | 0.01 | −5.84 | 1.32 | | | | | | | | |
| phenanthrene | −3.88 | −3.97 | 0.09 | −5.85 | 1.97 | | | | | | | | |
| Biphenyl | −2.66 | −2.79 | 0.13 | −5.30 | 2.64 | | | | | | | −3.1 | 0.31 |
| Toluene | −0.89 | −0.99 | 0.10 | −3.30 | 2.41 | −0.30 | 0.59 | −1.60 | 0.71 | | | −0.87 | 0.12 |
| Phenol | −6.62 | −6.80 | 0.18 | −7.54 | 0.92 | −5.20 | 1.42 | −4.00 | 2.62 | | | −6.18 | 0.62 |
| p-cresol | −6.14 | −6.71 | 0.57 | −9.06 | 2.92 | −4.80 | 1.34 | −3.90 | 2.24 | −6.09 | 0.05 | | |
| m-cresol | −5.49 | −6.52 | 1.03 | −9.46 | 3.97 | −4.80 | 0.69 | −4.00 | 1.49 | −6.21 | 0.72 | | |
| o-cresol | −5.87 | −6.15 | 0.28 | −7.08 | 1.21 | −4.60 | 1.27 | −3.70 | 2.17 | −6.06 | 0.19 | | |
| Water | −6.30 | −6.13 | 0.17 | −8.59 | 2.29 | −8.00 | 1.70 | −4.70 | 1.60 | | | −8.66 | 2.53 |
| Alcohols | | | | | | | | | | | | | |
| Methanol | −5.11 | −4.76 | 0.35 | −6.48 | 1.37 | −4.20 | 0.91 | −3.20 | 1.91 | | | −4.94 | 0.18 |
| Ethanol | −5.01 | −4.94 | 0.07 | −6.20 | 1.19 | −4.20 | 0.81 | −3.10 | 1.91 | −5.09 | 0.08 | −4.87 | 0.07 |
| Propanol | −4.92 | −4.74 | 0.18 | −6.22 | 1.30 | −4.10 | 0.82 | −3.10 | 1.82 | −5.03 | 0.11 | −4.62 | 0.12 |
| 2-Propanol | −4.62 | −4.62 | 0.00 | −6.27 | 1.65 | −4.30 | 0.32 | −3.10 | 1.52 | −4.33 | 0.29 | −4.62 | 0.00 |
| 1-Butanol | −4.72 | −4.66 | 0.06 | −6.55 | 1.83 | −3.90 | 0.82 | −3.10 | 1.62 | −4.72 | 0.00 | −4.56 | 0.10 |
| 2-Butanol | −4.58 | −4.66 | 0.08 | −5.69 | 1.11 | | | | | −3.97 | 0.61 | −3.89 | 0.77 |
| 2-Methyl-2- | −4.51 | −4.16 | 0.35 | −5.82 | 1.31 | −3.80 | 0.71 | −3.00 | 1.51 | | | −4.26 | 0.10 |
| Propanol | | | | | | | | | | | | | |
| 1-Pentanol | −4.47 | −4.52 | 0.05 | −6.68 | 2.21 | −4.00 | 0.47 | −3.30 | 1.17 | −4.77 | 0.30 | −4.41 | 0.11 |
| 2-Methyl-2- | −4.43 | −4.60 | 0.17 | −5.59 | 1.16 | | | | | | | | |
| Butanol | | | | | | | | | | | | | |
| 2-Pentanol | −4.39 | −4.47 | 0.08 | −5.77 | 1.38 | | | | | −3.99 | 0.40 | | |
| 3-Pentanol | −4.35 | −4.20 | 0.15 | −5.36 | 1.01 | | | | | −3.77 | 0.58 | | |
| Cyclohexanol | −5.47 | −5.13 | 0.34 | −6.50 | 1.03 | | | | | | | −5.46 | 0.33 |
| Octanol | −4.09 | −3.87 | 0.22 | −6.66 | 2.57 | −3.40 | 0.69 | −3.30 | 0.79 | −4.07 | 0.02 | −4.05 | 0.18 |
| Esters | | | | | | | | | | | | | |
| EthylAcetate | −3.12 | −2.80 | 0.32 | −7.07 | 3.95 | −3.10 | 0.02 | −3.70 | 0.58 | −3.10 | 0.02 | | |
| MethylPropionate | −2.93 | −3.03 | 0.10 | −6.68 | 3.75 | −2.70 | 0.23 | −3.50 | 0.57 | −3.13 | 0.20 | | |
| PropylAcetate | −2.86 | −2.79 | 0.07 | −6.98 | 4.12 | −3.00 | 0.14 | −3.80 | 0.94 | −3.10 | 0.24 | | |
| ButylAcetate | −2.55 | −2.48 | 0.07 | −6.74 | 4.19 | −2.90 | 0.35 | −3.80 | 1.25 | −3.16 | 0.61 | | |
| Amides | | | | | | | | | | | | | |
| Acetamide | −9.71 | −9.65 | 0.06 | −13.82 | 4.11 | −9.00 | 0.71 | −6.90 | 2.81 | | | −12.2 | 2.55 |
| N- | −10.00 | −10.49 | 0.49 | −13.10 | 3.10 | −8.60 | 1.40 | −6.60 | 3.40 | −8.11 | 1.89 | −10.22 | 0.27 |
| Methylacetamide | | | | | | | | | | | | | |
| DiMethylAcetamide | −8.50 | −8.51 | 0.01 | −10.81 | 2.31 | | | | | | | −8.14 | 0.37 |
| propionamide | −9.40 | −9.57 | 0.17 | −12.90 | 3.50 | | | | | −7.19 | 2.21 | | |
| Thiols | | | | | | | | | | | | | |
| MethaneThiol | −1.22 | −1.65 | 0.43 | −3.93 | 2.71 | −0.80 | 0.42 | −2.10 | 0.88 | | | | |
| EthaneThiol | −1.24 | −1.67 | 0.43 | −3.94 | 2.70 | −1.00 | 0.24 | −2.10 | 0.86 | | | | |
| Propanethiol | −1.05 | −1.45 | 0.40 | −3.93 | 2.88 | −0.90 | 0.15 | −2.20 | 1.15 | | | | |
| 1-Butanethiol | −0.99 | −1.49 | 0.50 | −4.05 | 3.06 | | | | | | | | |
| Sulfides | | | | | | | | | | | | | |
| DiMethylSulfide | −1.54 | −1.70 | 0.16 | −4.15 | 2.61 | −0.30 | 1.24 | −2.10 | 0.56 | | | | |
| DiEthylSulfide | −1.43 | −1.55 | 0.12 | −4.80 | 3.37 | −0.20 | 1.23 | −2.00 | 0.57 | | | | |
| EthylMethylSulfide | −1.50 | −1.61 | 0.11 | −4.20 | 2.70 | | | | | | | | |
| DimethylDisulfide | −1.83 | −1.85 | 0.02 | −4.67 | 2.84 | −1.10 | 0.73 | −2.40 | 0.57 | | | | |
| Heterorings | | | | | | | | | | | | | |
| 3-methyl-indole | −5.88 | −6.90 | 1.02 | −7.83 | 1.95 | | | | | | | −5.91 | 0.99 |
| Aniline | −5.54 | −5.48 | 0.06 | −7.67 | 2.13 | −4.80 | 0.74 | −4.10 | 1.44 | | | −5.71 | 0.23 |
| Imidazole | −9.60 | −9.89 | 0.29 | −13.31 | 3.71 | −9.00 | 0.60 | −6.70 | 2.90 | | | −11.19 | 1.30 |
| Pyrrole | −4.80 | −4.17 | 0.63 | −7.22 | 2.42 | | | | | −7.02 | 2.22 | −5.13 | 0.96 |
| pyridine | −4.69 | −4.77 | 0.08 | −6.73 | 2.04 | −4.20 | 0.49 | −3.20 | 1.49 | −4.90 | 0.21 | −4.04 | 0.73 |
| MAE | | | 0.20 | | 2.50 | | 0.61 | | 1.49 | | 0.50 | | 0.42 |

### Applications

The QM-parameterized force field models and the QM-parameterized force field molecular simulations described herein may be applied to a variety of molecular systems. Example applications include, but are not limited to, protein-ligand related applications and material science related applications. Example protein-ligand related applications can include, but are not limited to, protein-protein interactions related applications, protein-ligand interactions related applications, protein enzyme related applications, ligand properties related applications, membrane and metalloprotein applications, protein folding and various protein properties applications.

Embodiments can include performing free energy perturbation (FEP) calculations to predict ligand-protein binding affinities via molecular simulations using the force-field model.

Embodiments can include using the force-field model to screen drug candidate options through assessment of a plurality of variations of the drug candidate obtained by replacement of a central core of the drug candidate molecule with a plurality of chemical moieties to identify an optimum and/or potential drug candidate.

Embodiments can include using the force-field model in assessing ligand and protein targets in a lead optimization phase of drug development.

Embodiments can include using the force-field model to design ligands that bind to a protein for treatment of a disease.

Embodiments can include application to aggregation of biotherapeutic formulations.

Embodiments can include using the force-field model in predicting aggregation of a biotherapeutic including an antibody, wherein the predicting includes: predicting chromatography retention times of the antibody modeled with the force-field model; mapping distribution of aggregation-prone regions on a surface of the antibody modeled by the force-field model; and/or predicting effect of residue mutation on aggregation behavior of the antibody modeled by the force-field model.

Embodiments can include predicting structure of antibodies, in particular, using the force-field model to predict the three-dimensional structures of antibodies.

Embodiments can include using the force-field model to predict protein-protein binding.

Embodiments can include using the force-field model to design a protein having a stabilized folded protein conformation through modeling structural modifications of the protein.

Embodiments can include using the force-field model to design an enzyme through modeling of active variants of an enzyme.

Example material science related applications (homogenous and heterogenous liquids or simple structured systems like crystals, etc.) can include batteries, polymers, zeolites, solvents and mixtures and phase diagrams, and/or the like.

Embodiments can include using the force-field model to enhance reactivity and selectivity of transition metal catalyzed reactions through modification of one or more of the catalyst, catalyst substrate, and reactants modeled by the force-field model.

Embodiments can include using the force-field model to predict internal optoelectronic and condensed-phase thermophysical properties of organic optoelectronic materials in applications to optimize and/or improve material behavior. The material behavior can be selected from the group including charge injection, charge transport, exciton coupling, phosphorescence, chemical stability, thermophysical stability, etc. The applications can be selected from the group including organic light-emitting diodes (OLEDs), organic field-effect transistors (OFETs), photovoltaics (PVs and OPVs), dye-sensitized solar cells (DSSCs), etc.

Embodiments can include using the force-field model to predict controlled release of a drug from formulations, wherein the formulations include lipid or polymer-based aggregates in solution that sequester, solubilize and deliver the drug. A simulation of the formulation modeled by the force-field model predicts formation and structure of the aggregates and location of the drug within the aggregate.

Embodiments can include using the force-field model to predict stability of a drug compound to degradation mechanisms comprising oxidation, hydrolysis, photo-degradation, or any combination thereof. Degradation analyses over multiple chemical moieties in high-throughput fashion may provide useful criteria for rapid screening of formulation scenarios.

Embodiments may include using the force-field model to predict miscibility of a pharmaceutical formulation in a solution.

Embodiments may include using the force-field model to predict a glass transition temperature (Tg) of an amorphous solid obtained from density-temperature curves generated from simulations of a force-field model of the amorphous solid.

Applications may include those related to energy capture and storage. Embodiments may include modeling materials for batteries, fuel cells, and/or hydrogen storage using the force-field model to predict properties selected from the group including, for example, ion mobility, intercalation potentials and load capacity of battery electrodes, additives chemistry at an electrode-electrolyte interface, electro-catalytic activity, degradation processes, elastic and thermophysical properties, and/or ionic mobility in organic and solid electrolyte.

Embodiments may include modeling atomic layer deposition at a gas-surface interface using the force-field model.

Embodiments may include modeling organometallic complexes as precursors for depositing metal or metal oxide film at a gas-surface interface using the force-field model. A plurality of variations of organometallic complexes may be screened to assess viability of organometallic complexes as precursors. The variations may include a collection of complexes having different combinations of selected ligands associated with a metal ion.

Embodiments may include modeling polymer systems using the force-field model to determine mechanical and thermophysical properties, wherein the polymer system can be selected from the group including a crosslinked polymer, a carbon fiber polymer composite, a polymer-polymer composite, a polymer-ceramic composite, a neat polymer and/or the like.

Embodiments may include determining elastic and ultimate performance properties of a polymer system modeled with the force-field model. The polymer system model may be subjected to a series of simulations under different levels of a controlled strain and a stress calculated from the simulations and the strain may be used to determine modulus and a yield point of the polymer system.

Embodiments may include determining a glass transition temperature (Tg) of a polymer system modeled with the force-field model, wherein a series of simulations of the polymer system may be performed at different temperatures and the Tg is determined from a glass transition identified from the densities calculated from the simulations and the temperatures.

Embodiments may include assessing miscibility of components in a polymer material through modeling with the force-field model, wherein the components can include reactive components, solvents, polymer additives, plasticizers, or any combination thereof. Assessing miscibility may include comparing solubility parameters determined from the modeling.

Embodiments include modeling phase separation in polymer blends and copolymer systems with the force-field model.

Embodiments include modeling inorganic solid materials with the force-field model to predict bulk and surface properties, wherein the properties can be selected from the group including band structure, mechanical, dielectric, magnetic, and/or thermodynamic properties.

Embodiments may include predicting compositional phase diagrams, material defects, and/or surface degradation and reactivity of the inorganic solid materials modeled with the force-field model.

Embodiments may include modeling membrane proteins with the force-field model including molecular dynamics simulations of protein-protein interactions and protein-lipid interactions.

Embodiments can include performing a molecular simulation of the force-field model of a metalloprotein in a biological system. A relationship between structure and function of the metalloprotein as a catalyst for use in synthesis of model catalysts that mimic the modeled metalloprotein may be evaluated from molecular simulation.

Embodiments may include performing a molecular simulation of permeation of compounds through a skin barrier layer using the force-field model to predict permeability of the compounds through the skin barrier layer. The compounds may be selected from the group including drugs, toxic chemicals, and/or cosmetics.

Embodiments may include molecular simulation of misfolding and aggregation of proteins modeled with the force-field model to elucidate conformational states and the aggregation and the misfolding pathways of the proteins.

Embodiments may include performing molecular simulations of polymer nanocomposites (PNCs) modeled as organic or inorganic nanoparticles interacting with polymer chains represented with the force-field model, wherein a conformation and the mobility of the polymer chains in proximity to the nanoparticles is monitored. A relationship of enthalpic and entropic interactions on structure and the dynamics of the polymer chains and the nanoparticles to macroscopic properties may be evaluated from the simulations.

Methods described herein can further include performing molecular simulations of a liquid crystal system using the force-field model, wherein the liquid system is selected from the group including a bulk phase, mesophase, inhomogeneous system, interfacial system, etc.

Embodiments may include performing molecular simulations of adsorption, separation and/or diffusion of molecular systems in a zeolite. The molecular system is selected from the group including small molecules comprising less than 10 atoms, long chain hydrocarbons including greater than 10 carbons, mixtures, etc. The results of the simulations may be used to evaluate and/or calculate adsorption isotherms of molecules, separation of molecules of different types, and relative diffusion of molecules of different types of the molecular system.

Embodiments may include simulations that probe protein-lipid interactions of single membrane proteins and complex membranes.

Embodiments may include molecular simulation that can be used to study the relationship between structure and function of metalloproteins, which can be used to synthesize model catalysts that mimic those metalloproteins.

Embodiments may include molecular simulations used to understand and predict permeability of compounds through skin. This is of interest for transdermal delivery of drugs, for toxicity predictions of chemicals and topical application of cosmetics. Permeability of compounds can be simulated using atomistic models of the skin's barrier structure and the compounds. Modeling predictions may be validated with data for reference compounds.

Embodiments may include using MD simulations with the force field described herein to provide a full and realistic dynamical description of the protein-ligand binding event.

Embodiments may include determining functional mechanisms of proteins through simulation of transitions of a protein from an active to inactive structure. The atomistic characterization of the transition state is a fundamental step to improve the understanding of the folding mechanism and the function of proteins.

Embodiments may include using the force field model described herein to understand the optical, electrical and mechanical properties of organic electronic materials. The spatial arrangement of the molecules as well as the chemical identity critically affects the performance of the material. Modeling can be used to relate a material's performance in a device to the molecular details that cause and optimize that performance. Processes such as charge transport, exciton coupling, and phosphorescence are important for optimizing and/or improving the performance of materials used in organic electronics devices such as organic light-emitting diodes (OLEDs), organic field-effect transistors (OFETs), photovoltaics (PVs and OPVs), and dye-sensitized solar cells (DSSCs).

Polymer nanocomposites (PNCs) are hybrid materials incorporating organic or inorganic nanoparticles (NPs) with at least one dimension in the submicron scale. These materials play an important role in industrial formulations and technological applications from food packaging to smart coatings. Incorporating nanoparticles (NPs) to a polymer matrix can significantly alter the conformation and the mobility of the polymer chains in their proximity. Understanding the delicate balance between the enthalpic and entropic interactions is crucial to control and predict the ability of NPs to diffuse and disperse in the polymer matrix. The relationship of these interactions on the structure and the dynamics of polymer chains and NPs to a number of macroscopic properties can be shown by molecular dynamics simulations using the force field described herein.

Molecular dynamics simulations may be employed to study liquid crystals including bulk phases, mesophases, inhomogeneous systems, and interfaces. In self-organizing materials, such as liquid crystals, subtle changes in intermolecular forces lead to changes in phase behavior. In liquid crystals many properties of interest arise from specific ordering of molecules (or parts of molecules) in the bulk and so are studied by simulation of many molecules.

Molecular simulation has been applied to study adsorption, separation and diffusion of various types of molecules in zeolites. There are numerous industrial applications involving pure components, various sizes of molecules including small molecules to long chain hydrocarbons, and mixtures. MD simulations can relate the adsorbates' characteristics to adsorption isotherms, separation, and relative diffusion.

It should be appreciated that the above detailed embodiments of the present disclosure are only to exemplify or explain principles of the present disclosure and not to limit the present disclosure. Therefore, any modifications, equivalent alternatives and improvement, etc. without departing from the scope of the present disclosure shall be included in the scope of protection of the present disclosure. Meanwhile, appended claims of the present disclosure aim to cover all the variations and modifications falling under the scope and boundary of the claims or equivalents of the scope and boundary.

The above-described embodiments can be implemented in any of numerous ways. For example, embodiments may be implemented using hardware, software or a combination thereof. When implemented in software, the software code can be stored (e.g., on non-transitory memory) and executed on any suitable processor or collection of processors, whether provided in a single computer or distributed among multiple computers.

Further, it should be appreciated that a compute device including a computer can be embodied in any of a number of forms, such as a rack-mounted computer, a desktop computer, a laptop computer, netbook computer, or a tablet computer. Additionally, a computer can be embedded in a device not generally regarded as a computer but with suitable processing capabilities, including a smartphone, smart device, or any other suitable portable or fixed electronic device.

Also, a computer can have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer can receive input information through speech recognition or in other audible format.

Such computers can be interconnected by one or more networks in any suitable form, including a local area network or a wide area network, such as an enterprise network, and intelligent network (IN) or the Internet. Such networks can be based on any suitable technology and can operate according to any suitable protocol and can include wireless networks, wired networks or fiber optic networks.

The various methods or processes outlined herein can be coded as software that is executable on one or more processors that employ any one of a variety of operating systems or platforms. Additionally, such software can be written using any of a number of suitable programming languages and/or programming or scripting tools, and also can be compiled as executable machine language code or intermediate code that is executed on a framework or virtual machine.

In this respect, various disclosed concepts can be embodied as a computer-readable storage medium (or multiple computer-readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer-readable medium or media can be transportable, such that the program or programs stored thereon can be loaded onto one or more different computers or other processors to implement various aspects of the present disclosure as discussed above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

The terms “program” or “software” or “software stack” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but can be distributed in a modular fashion amongst a number of different computers or processors to implement various aspects of the disclosure.

Computer-executable instructions can be in many forms, such as program modules, executed by one or more computers or other devices. Generally, program modules include routines, programs, objects, components, data structures, etc. that perform particular tasks or implement particular abstract data types. Typically the functionality of the program modules can be combined or distributed as desired in various embodiments.

Also, various concepts can be embodied as one or more methods, of which an example has been provided. The acts performed as part of the method may be ordered in any suitable way. Accordingly, embodiments can be constructed in which acts are performed in an order different than illustrated, which can include performing some acts simultaneously, even though shown as sequential acts in illustrative embodiments. All publications, patent applications, patents, and other references mentioned herein are incorporated by reference in their entirety.

What is claimed is:

1. A method, comprising:
determining, by a processor, an atom type from a plurality of atom types of each atom from a plurality of atoms in a functional group of a molecular system;
calculating, by the processor and based on the plurality of atom types, a first plurality of ab initio molecular properties of a monomer having the functional group;
calculating, by the processor and based on the plurality of atom types, a second plurality of ab initio molecular properties of a multimer having the functional group;
determining, by the processor, a plurality of parameters of a force field model by fitting the force field model to the first plurality of ab initio molecular properties and the second plurality of ab initio molecular properties until at least one of (1) a first difference between a first plurality of predicted molecular properties of the monomer and the first plurality of ab initio molecular properties of the monomer reaches a pre-determined threshold or (2) a second difference between a second plurality of predicted molecular properties of the multimer and the second plurality of ab initio molecular properties of the multimer reaches the pre-determined threshold, the force field model describing intermolecular interactions and intramolecular interactions of the molecular system;
determining, by the processor and based on the plurality of parameters and the force field model, a molecular property of the molecular system; and
sending, by the processor, a signal to present the molecular property of the molecular system.

2. The method of claim 1, wherein the molecular system includes at least one of homogeneous liquids, a heterogeneous mixture, liquid crystals, or a ligand-protein system.

3. The method of claim 1, wherein the force field model is transferable to a different molecular system from the molecular system such that molecular properties of the different molecular system predicted based on the force field model is within a chemical accuracy of the molecular properties of the different molecular system determined from experiment.

4. The method of claim 1, further comprising:
selecting the functional group of the molecule to include, in the functional group, at least one of carbon (C), oxygen (O), nitrogen (N), hydrogen (H), phosphorus (P), sulphur (S), chlorine (Cl), fluorine, bromine, or iodine.

5. The method of claim 1, further comprising:
selecting the functional group of the molecule to include, in the functional group, at least one of an aromatic carbon, an alkane carbon, an amide nitrogen, a hydroxyl oxygen, a methyl carbon, a carbonyl carbon, a carboxyl carbon, an amino nitrogen, a phosphate phosphorus, or a sulfhydryl sulphur.

6. The method of claim 1, wherein:
the determining the atom type of the atom is based on at least one of:
(1) a chemical identity of the atom determined by a number of bound electrons of the atom;
(2) properties of a molecular neighborhood of the atom including chemical identities, arrangement and hybridization of a set of atoms neighboring the atom and atom types of each atom from the set of atoms, the set of atoms included in the plurality of atoms; or
(c) at least one characteristic of the atom including shape or density of an electronic structure of the atom, effective dispersion coefficients, polarization coefficients, or a location of the atom relative to the set of atoms neighboring the atom.

7. The method of claim 6, wherein the shape and the density of the electronic structure of the atom include at least one of a dipole moment, a quadrupole moment, or a multipole moment.

8. The method of claim 1, wherein:
the first plurality of ab initio molecular properties of the monomer includes at least one of molecular multipole moment, molecular electrostatic field, energy of molecular conformation, or polarization by an applied external field or charge.

9. The method of claim 1, wherein the determining the molecular property is based on thermodynamic sampling of the molecular system, the thermodynamic sampling of the molecular system being based on at least one of molecular dynamics simulation, stochastic simulation, Monte-Carlo simulation, or path integral molecular dynamics (PIMD).

10. The method of claim 1, wherein the determining the molecular property of the molecular system is based on thermodynamic sampling and nuclear quantum effects (NQE).

11. The method of claim 1, wherein the second plurality of ab initio molecular properties of the multimer include at least one of:
potential energy of hetero-multimer interactions between the monomer and a first probe species,
potential energy of homo-multimer interactions between two identical molecules of the monomer, or
potential energy of multimer-multimer interactions between the monomer and at least two probe species having a second probe species and a third probe species,
the first probe species, the second probe species and the third probe species include at least one of a charge, a noble gas atom, or a small molecule.

12. The method of claim 11, wherein the small molecule includes at least one of a water molecule, a methane molecule, or an ammonia molecule.

13. The method of claim 11, wherein the potential energy of the hetero-multimer interactions, the potential anergy of the homo-multimer interactions, and the potential energy of the multimer-multimer interactions are calculated based on a plurality of orientations of the hetero-multimer interactions, the homo-multimer interactions, and the multimer-multimer interactions, respectively.

14. The method of claim 11, wherein the potential energy of the hetero-multimer interactions, the potential anergy of homo-multimer interactions, and the potential energy of multimer-multimer interactions are calculated based on a plurality of distances of the hetero-multimer interactions, the homo-multimer interactions, and the multimer-multimer interactions, respectively.

15. The method of claim 1, wherein the fitting the force field model to the first plurality of ab initio molecular properties and the second plurality of ab initio molecular properties includes adjusting at least one parameter from the plurality of parameters of the force field model to cause electrostatic potential of the monomer determined using the force field model to be in a pre-determined range of electrostatic potential of the monomer from the first plurality of ab initio molecular properties.

16. The method of claim 1, wherein the fitting the force field model to the first plurality of ab initio molecular properties and the second plurality of ab initio molecular properties includes adjusting a polarization parameter from the plurality of parameters of the force field model or an induction parameter from the plurality of parameters to produce an effect of a probe charge or an external electric field placed around the monomer.

17. The method of claim 1, wherein the fitting the force field model to the first plurality of ab initio molecular properties and the second plurality of ab initio molecular properties includes adjusting a polarization parameter from the plurality of parameters of the force field model or an induction parameter from the plurality of parameters to cause an induction component of dimerization energy of the multimer determined using the force field model to be in a pre-determined range of the induction component of the multimer from the second plurality of ab initio molecular properties.

18. The method of claim 1, wherein the force field model includes a plurality of components associated with at least one of exchange energy, dispersion energy, electrostatics energy, charge transfer interaction, induction energy, exchange-induction energy, penetration effect in electrostatics potential, damping effect in dispersion energy, multipolar electrostatics energy, multipolar exchange energy, multipolar dispersion interaction energy, or many-body interaction energy.

19. The method of claim 1, wherein the molecular property includes at least one of density, heat of vaporization, heat of melting, heat of sublimation, free energy of solvation, or free energy of ligand binding in ligand-protein systems, thermal conductivity, diffusion coefficient, X-ray diffraction, glass transition temperature, glass transition enthalpy change, or enthalpy of solid-solid phase transitions.

20. The method of claim 1, wherein the intermolecular interactions of the molecular system include an electric field.

21. The method of claim 1, wherein the force field model is associated with auxiliary interaction locations including an electron lone pair site or mid-bond site.

22. The method of claim 1, wherein the pre-determined threshold is a chemical accuracy associated with thermal noise. at room temperature and pressure, substantially being 0.59 kcal/mol or kT at 300K.

23. The method of claim 1, wherein determining the molecular property to identify a drug candidate.

24. The method of claim 1, wherein determining the molecular property to design ligands that bind to a protein for treatment of a disease.

25. The method of claim 1, wherein the multimer is a molecule that includes two or more monomers.

26. The method of claim 1, wherein the multimer is a dimer, a trimer, or a tetramer.

27. An apparatus, comprising:
a memory for storing a force field model including information of intermolecular interactions and intramolecular interactions of a molecular system;
a processor operatively coupled to the memory, the processor configured to:
determine an atom type from a plurality of atom types of each atom from a plurality of atoms in a functional group of the molecular system;
calculate, based on the plurality of atom types, a plurality of ab initio molecular properties of at least one of a monomer having the functional group or a multimer having the functional group;
determine a plurality of parameters of the force field model by fitting the force field model to the plurality of ab initio molecular properties until a difference between a plurality of predicted molecular properties and the plurality of ab initio molecular properties reaches a pre-determined threshold;
determine, based on the plurality of parameters and the force field model, a molecular property of the molecular system, the force field model being transferable to a different molecular system from the molecular system such that molecular properties of the different molecular system predicted based on the force field model is within a chemical accuracy of the molecular properties of the different molecular system determined from experiment; and
send a signal to present the molecular property of the molecular system.

28. A processor-readable non-transitory medium storing code representing instructions to be executed by a processor, the code comprising code to cause the processor to:
determine an atom type from a plurality of atom types of each atom from a plurality of atoms in a functional group of a molecular system;
calculate, based on the plurality of atom types, a plurality of ab initio molecular properties of at least one of a monomer having the functional group or a multimer having the functional group;
determine a plurality of parameters of a force field model by fitting the force field model to the plurality of ab initio molecular properties until a difference between a plurality of predicted molecular properties and the plurality of ab initio molecular properties reaches a pre-determined threshold, the force field model including information of intermolecular interactions and intramolecular interactions of the molecular system;
determining, based on the plurality of parameters and the force field model, a molecular property of the molecular system to identify a drug candidate; and
sending a signal to present the molecular property of the molecular system.
